(19) United States
(12) Patent Application Publication
(10) Pub. No.: US 2012/0283115 A1
(43) Pub. Date: Nov. 8, 2012

(54) SEROMIC ANALYSIS OF OVARIAN CANCER

(75) Inventors: Gerd Ritter, New York, NY (US); Lloyd J. Old, New York, NY (US); Constance Old, New York, NY (US); Sacha Gnjatic, New York, NY (US); Kunle Odunsi, Buffalo, NY (US); Dirk Jager, Heidelberg (DE)

(73) Assignee: Ludwig Institute for Cancer Research Ltd., New York, NY (US)

(21) Appl. No.: 13/392,919

(22) PCT Filed: Aug. 30, 2010
(86) PCT No.: PCT/US10/02374

§ 371 Date: Jul. 27, 2012

Related U.S. Application Data
(60) Provisional application No. 61/238,377, filed on Aug. 31, 2009.

(51) Int. Cl. G01N 33/574; G01N 33/82; C40B 30/04; G01N 21/64; B82Y 15/00

(52) U.S. Cl. 506/9; 436/501; 435/7.92; 977/774; 977/920

(57) ABSTRACT

The invention relates to the discovery of cancer antigens associated with ovarian cancer. In further aspects, the invention relates to methods, compositions and kits for the diagnosis and treatment of cancer, particularly ovarian and pancreatic cancers.

### RELATED APPLICATIONS

This application claims the benefit of the filing date under 35 USC §119 of U.S. provisional application 61/238,377 filed on Aug. 31, 2009, the entire contents of which are incorporated herein by reference.

### GOVERNMENT SUPPORT

The invention was made with Government support through National Cancer Institute Center grant CA 16056. The Government has certain rights in the invention.

### FIELD OF THE INVENTION

The invention relates to the discovery of cancer antigens associated with ovarian cancer. In further aspects, the invention relates to methods, compositions and kits for the diagnosis, prognosis and treatment of cancer, particularly ovarian cancer.

### BACKGROUND OF THE INVENTION

Studies of the cellular and humoral immune response to cancer have revealed a repertoire of antigens recognized by the immune system, collectively termed the cancer immunome. Moreover, prolonged survival in certain individuals with cancer has been associated with natural antibodies that bind cancer antigens (Livingston P O, et al., J Clin Oncol 12:1036-1044, 1994; Jones P C, et al, J Natl Cancer Inst 66:249-254, 1981; and Graus F, et al; J Clin Oncol 15:2866-2872, 1997). The identification of such cancer antigens that are recognized by the autologous host is yielding new and promising target molecules for immunotherapy, diagnosis and monitoring of human cancer.

Ovarian cancer is the leading cause of death in women with gynecological malignancies (Greenlee R T et al., Cancer statistics, 2001. CA Cancer J. Clin. (2001) 51(1):15-36). Identification of new cancer antigens is needed for the development of additional therapeutics and diagnostics to permit effective treatment and diagnosis of patients suffering from ovarian cancer.

### SUMMARY OF THE INVENTION

Some aspects of this invention relate to the discovery of a series of antigens recognized by autoantibodies present in the serum of ovarian cancer patients. Autoantibody antigens are disclosed herein that are useful either individually or as signature sets as, for example, (i) diagnostic markers, (ii) prognostic markers associated with favorable or unfavorable clinical outcome, and/or (iii) potential targets of immune responses for the development of new immunotherapeutic reagents.

Methods, compositions and kits useful for the diagnosis, prognosis, prevention, and treatment of cancer based on newly discovered cancer antigens are described herein. In some aspects, cancer antigens are disclosed that were identified in human serum samples obtained from patients having ovarian or pancreatic cancer. The antigens were identified through the use of protein microarrays (polypeptide arrays) containing thousands of full-length protein antigens. The arrays were used to detect specific serum antibody responses in cancer patients. A set of 197 proteins were identified as being immunogenic in ovarian cancer patients (see Table 3). These cancer antigens are useful for both diagnostic and therapeutic purposes.

According to some aspects of the invention, methods for diagnosing ovarian cancer in a human are provided. The methods involve contacting a biological sample obtained from the human with at least one polypeptide selected from the polypeptides encoded by the transcripts disclosed in Table 3; and determining an amount of specific binding between the at least one polypeptide and at least one antibody in the biological sample, wherein the amount of specific binding is diagnostic for ovarian cancer in the human.

In some embodiments, a method for diagnosis of ovarian cancer in a subject I provided, the method comprising (a) obtaining a biological sample from the subject; (b) contacting the biological sample with a polypeptide selected from the polypeptides encoded by a transcript disclosed in Table 3, or with an antibody-binding fragment of the polypeptide; and (c) determining the absence or the presence of an autoantibody specifically binding the polypeptide in the biological sample; wherein (i) the presence of an autoantibody specifically binding a peptide encoded by a transcript disclosed in Table 3 in the biological sample is indicative of the subject having ovarian cancer, and/or (ii) the absence of an autoantibody specifically binding a peptide encoded by a transcript disclosed in Table 3 in the biological sample is indicative of the subject not having ovarian cancer; and/or (d) determining a level of an autoantibody specifically binding the polypeptide in the biological sample and comparing the level to a reference or control level, wherein, (i) an elevated level of an autoantibody specifically binding a peptide encoded by a transcript disclosed in Table 3 in the biological sample as compared to the reference or control level is indicative of the subject having ovarian cancer, and/or (ii) a non-elevated level of an autoantibody specifically binding a peptide encoded by a transcript disclosed in Table 3 in the biological sample as compared to the reference or control level is indicative of the subject not having ovarian cancer.

In some embodiments, the presence or an elevated level of an autoantibody specifically binding a peptide encoded by a transcript selected from the group consisting of ANXA2, FAM1318, FER, ZIM2, and NY-ESO1 transcripts is indicative of an increased expected survival time of the subject as compared to the average survival time of subjects having ovarian cancer or to the average survival time of ovarian cancer subjects in which the autoantibody is absent. In some embodiments, the presence or elevated levels of autoantibodies specifically binding peptides encoded by ANXA2, FAM1318, FER, and ZIM2 transcripts is/are indicative of an increased expected survival time of the patient as compared to the average survival time of ovarian cancer patients or to the average survival time of ovarian cancer subjects in which the autoantibody is absent. In some embodiments, the presence or an elevated level of an autoantibody specifically binding a peptide encoded by a transcript selected from the group consisting of ERFI1, PHLDB1, TRH, TRUB1, UBTD2 (DC-UbP) transcripts is indicative of a decreased expected survival time of the patient as compared to the average survival time of ovarian cancer patients or to the average survival time of ovarian cancer subjects in which the autoantibody is absent. In some embodiments, the presence or elevated levels of autoantibodies specifically binding peptides encoded by ERFI1, PHLDB1, TRH, and TRUB1 transcripts is/are indicative of a decreased expected survival time of the patient as compared to the average survival time of ovarian cancer patients or to the average survival time of ovarian cancer subjects in which the autoantibody is absent. In some embodiments, the biological sample is a blood sample.

In some embodiments, a method for prognosing patient outcome in ovarian cancer is provided, the method comprising determining the presence or a level of an autoantibody specifically binding a peptide encoded by a transcript selected from the group consisting of ANXA2, FAM1318, FER, ZIM2, NY-ESO1, ERFI1, PHLDB1, TRH, TRUB1, and UBTD2 (DC-UbP) transcripts in a subject having ovarian cancer, wherein the presence of an autoantibody specifically binding a peptide encoded by an ANXA2, FAM1318, FER, ZIM2, or NY-ESO1 transcript or an elevated level of said autoantibody as compared to a reference or control level is indicative of a better patient outcome as compared to the average patient outcome of ovarian cancer patients, and/or wherein the presence of an autoantibody specifically binding a peptide encoded by a transcript chosen from the group consisting of ERFI1, PHLDB1, TRH, TRUB1, UBTD2 (DC-UbP) transcripts or an elevated level of said autoantibody as compared to a reference or control level is indicative of a worse patient outcome as compared to the average survival time of ovarian cancer patients.

In some embodiments, the presence of an autoantibody specifically binding a peptide encoded by an ANXA2, FAM1318, FER, ZIM2, or NY-ESO1 transcript or an elevated level of said autoantibody as compared to a reference or control level is indicative of an increased expected survival time of the patient as compared to the average survival time of ovarian cancer patients, and/or wherein the presence of an autoantibody specifically binding a peptide encoded by a transcript chosen from the group consisting of ERFI1, PHLDB1, TRH, TRUB1, UBTD2 (DC-UbP) transcripts or an elevated level of said autoantibody as compared to a reference or control level is indicative of a decreased expected survival time of the patient as compared to the average survival time of ovarian cancer patients. In some embodiments, the presence or elevated levels of autoantibodies specifically binding peptides encoded by ANXA2, FAM1318, FER, and ZIM2 transcripts is/are indicative of an increased expected survival time of the patient as compared to the average survival time of ovarian cancer patients. In some embodiments, the presence or elevated levels of autoantibodies specifically binding peptides encoded by ERFI1, PHLDB1, TRH, and TRUB1 transcripts is/are indicative of a decreased expected survival time of the patient as compared to the average survival time of ovarian cancer patients. In some embodiments, determining the presence or a level of an autoantibody comprises obtaining a biological sample from the subject and contacting the biological sample with a polypeptide encoded by a transcript selected from the group consisting of ANXA2, FAM1318, FER, ZIM2, NY-ESO1, ERFI1, PHLDB1, TRH, TRUB1, and UBTD2 (DC-UbP) transcripts, or an antibody-binding fragment of said polypeptide. In some embodiments, the biological sample is a blood sample.

In some embodiments, determining the presence or the absence of the autoantibody comprises performing an enzyme-linked immunoassay (ELISA). In some embodiments, the polypeptide is fixed to a solid substrate. In some embodiments, the solid substrate forms or is comprised in a plate well, and, optionally, wherein the plate well is in a multi-well plate optionally having a number of wells selected from the group consisting of: 6, 12, 24, 96, 384, and 1536. In some embodiments, the solid substrate is a polypeptide array surface. In some embodiments, determining the presence or absence of the autoantibody comprises contacting the autoantibody binding the polypeptide with a detection agent. In some embodiments, the detection agent is a secondary antibody or antibody fragment, or an autoantibody-binding polypeptide conjugated to a detectable label. In some embodiments, the detectable label is selected from the group consisting of: a radioisotope, a fluorophore, a luminescent molecule, an enzyme, a biotin-moiety, an epitope tag, and a dye molecule. In some embodiments, the detectable label catalyzes a chemical or biochemical reaction resulting in luminescence. In some embodiments, the detectable label is an enzyme selected from a phosphatase and a peroxidase. In some embodiments, the detectable label is a fluorophore selected from the group consisting of FITC, TRITC, Cy3, Cy5, Alexa Fluorescent Dyes, and Quantum Dots.

In some embodiments of the diagnostic or prognostic methods disclosed herein, the biological sample is a blood sample.

In some embodiments of the diagnostic or prognostic methods disclosed herein, the at least one polypeptide is fixed to a solid substrate. In certain embodiments, the solid substrate is a plate well, optionally wherein the plate well is in a multi-well plate optionally having a number of wells selected from: 6, 12, 24, 96, 384, and 1536. In certain embodiments, the solid substrate is a polypeptide array surface.

In some embodiments of the diagnostic methods disclosed herein, the determining comprises using an Enzyme Linked Immunoassay (ELISA) to evaluate the amount of specific binding.

In some embodiments of the diagnostic methods disclosed herein, the determining comprises contacting the at least one antibody with at least one secondary antibody or antigen binding fragment that specifically binds the at least one antibody. In certain embodiments, the secondary antibody or antigen binding fragment is conjugated to a detectable label. In specific embodiments, the detectable label is selected from: a radioisotope, a fluorophore, a luminescent molecule, an enzyme, a biotin-moiety, an epitope tag, and a dye molecule. In other embodiments, the detectable label activates a chemiluminescent substrate. In other embodiments, the detectable label is an enzyme selected from a phosphatase and a peroxidase. In other embodiments, the detectable label is a fluorophore selected from: FITC, TRITC, Cy3, Cy5, Alexa Fluorescent Dyes, and Quantum Dots.

In some embodiments, the diagnostic or prognostic methods disclosed herein also involve comparing the amount of specific binding to a reference level, wherein an amount of specific binding that is higher than the reference level is indicative of ovarian cancer in the human, and producing a patient report and/or changing a patient record to indicate a diagnosis of ovarian cancer in the human. In specific embodiments, the reference level comprises at least one level of an antibody that specifically binds the polypeptide in a biological sample obtained from a cancer-free human. In specific embodiments, the reference level comprises at least one level of at least one antibody that specifically binds the polypeptide in a biological sample obtained from a human having ovarian cancer. In specific embodiments, the reference level comprises at least one level of at least one antibody that specifically binds the polypeptide in a biological sample obtained from a human having ovarian cancer.

According to some aspects of the invention, methods for treating a human having, or at risk of having, ovarian cancer are provided. The methods involve administering to the human an effective amount of a composition comprising: (i) at least one polypeptide and/or at least one immunogenic fragment thereof that is encoded by a transcript disclosed in Table 3; (ii) at least one vector that expresses at least one polypeptide and/or at least one immunogenic fragment thereof that is encoded by a transcript disclosed in Table 3; and/or (iii) at least one antibody that binds specifically to a polypeptide that is encoded by a transcript disclosed in Table 3, wherein administration of the at least one polypeptide and/or at least one immunogenic fragment thereof and at least one vector induces an immune response against the at least one polypeptide and/or at least one immunogenic fragment thereof.

In some embodiments of the foregoing treatment methods, the vector is a plasmid or a virus.

In some embodiments of the foregoing treatment methods, the composition comprises an adjuvant. In certain embodiments, the adjuvant is one or more saponins, GM-CSF, one or more interleukins and/or one or more immunostimulatory oligonucleotides.

In some embodiments, a method is provided comprising eliciting or amplifying an immune response in a subject having or being at risk of developing ovarian cancer by administering to the subject a peptide encoded by a transcript in Table 3, or an immunogenic fragment thereof.

According to some aspects of the invention, pharmaceutical compositions are provided. In some embodiments, the pharmaceutical compositions include at least one polypeptide and/or at least one immunogenic fragment thereof that is encoded by a transcript disclosed in Table 3, and/or at least one vector that expresses at least one polypeptide and/or at least one immunogenic fragment thereof that is encoded by a transcript disclosed in Table 3; and a pharmaceutically acceptable adjuvant. In some embodiments, the pharmaceutical compositions include at least two polypeptides and/or at least two immunogenic fragments thereof that is encoded by a transcript disclosed in Table 3, and/or at least two vectors, each of which expresses at least one polypeptide and/or at least one immunogenic fragment thereof that is encoded by a transcript disclosed in Table 3; and a pharmaceutically acceptable adjuvant. In some embodiments, the pharmaceutical compositions include at least three polypeptides and/or at least three immunogenic fragments thereof that is encoded by a transcript disclosed in Table 3, and/or at least three vectors, each of which expresses at least one polypeptide and/or at least one immunogenic fragment thereof that is encoded by a transcript disclosed in Table 3; and a pharmaceutically acceptable adjuvant. In some embodiments, the pharmaceutical compositions comprise at least four polypeptides and/or at least four immunogenic fragments thereof that is encoded by a transcript disclosed in Table 3, and/or at least four vectors, each of which expresses at least one polypeptide and/or at least one immunogenic fragment thereof that is encoded by a transcript disclosed in Table 3; and a pharmaceutically acceptable adjuvant. In some embodiments, the pharmaceutical compositions comprise at least five polypeptides and/or at least five immunogenic fragments thereof that is encoded by a transcript disclosed in Table 3, and/or at least five vectors, each of which expresses at least one polypeptide and/or at least one immunogenic fragment thereof that is encoded by a transcript disclosed in Table 3; and a pharmaceutically acceptable adjuvant.

In some embodiments, the pharmaceutical compositions include at least one antibody that binds specifically to a polypeptide that is encoded by a transcript disclosed in Table 3; and a pharmaceutically acceptable adjuvant. In certain embodiments, the at least one antibody is selected from: a monoclonal antibody, a chimeric antibody, a humanized antibody, and an antigen-binding fragment, optionally wherein the antigen-binding fragment is selected from: F(ab′)₂, F(ab′), F(ab), a single domain antibody, and single chain Fv.

In some embodiments, the pharmaceutical compositions include at least two antibodies, wherein each antibody specifically binds a polypeptide that is encoded by a transcript disclosed in Table 3; and a pharmaceutically acceptable carrier. In certain embodiments, the pharmaceutical compositions also include a pharmaceutically acceptable adjuvant. In certain embodiments, each of the at least two antibodies is selected from: a monoclonal antibody, a chimeric antibody, a humanized antibody, and an antigen-binding fragment, optionally wherein the antigen-binding fragment is selected from: F(ab′)₂, F(ab′), F(ab), a single domain antibody, and single chain Fv.

In some embodiments of the foregoing pharmaceutical compositions, the pharmaceutically acceptable adjuvant is one or more saponins, GM-CSF, one or more interleukins and/or one or more immunostimulatory oligonucleotides.

According to some aspects of the invention polypeptide arrays are provided.

In some embodiments, the polypeptide arrays consist essentially of at least two polypeptides fixed to a solid substrate, wherein the at least two polypeptides are encoded by at least two transcripts disclosed in Table 3. In some embodiments, the polypeptide arrays consist essentially of at least three polypeptides fixed to a solid substrate, wherein the at least three polypeptides are encoded by at least three transcripts disclosed in Table 3. In some embodiments, the polypeptide arrays consist essentially of at least four polypeptides fixed to a solid substrate, wherein the at least four polypeptides are encoded by at least four transcripts disclosed in Table 3. In some embodiments, the polypeptide arrays consist essentially of at least five polypeptides fixed to a solid substrate, wherein the at least five polypeptides are encoded by at least five transcripts disclosed in Table 3.

According to some aspects of the invention, kits for diagnosing ovarian cancer in a human are provided. In some embodiments, the kits include at least one container housing at least two polypeptides encoded by at least two transcripts disclosed in Table 3; and instructions for use of the polypeptides in the diagnosis of ovarian cancer.

In some embodiments of the foregoing diagnostic kits, the solid substrate is a plate well, optionally wherein the plate well is in a multi-well plate optionally having a number of wells selected from: 6, 12, 24, 96, 384, and 1536. In one embodiment, the solid substrate is a polypeptide array surface.

According to some aspects of the invention, kits for vaccinating a human against ovarian cancer are provided. In some embodiments, the kits include at least one container housing a cancer vaccine comprising at least one polypeptide or an immunogenic fragment thereof that is encoded by a transcript disclosed in Table 3 and optionally a pharmaceutically acceptable adjuvant; and instructions for the administering the ovarian cancer vaccine to the human. In some embodiments, the kits include at least one container housing a composition comprising at least one antibody or antigen binding fragment that binds specifically to a polypeptide, or an immunogenic fragment thereof, that is encoded by a transcript disclosed in Table 3 and optionally a pharmaceutically acceptable adjuvant; and instructions for the administering the ovarian cancer vaccine to the human. In certain embodiments, the antibody or antigen binding fragment is selected from: a monoclonal antibody, a chimeric antibody, a humanized antibody, a F(ab′)₂ fragment, a F(ab′) fragment, a F(ab) fragment, a single domain antibody, and a single chain Fv.

### BRIEF DESCRIPTION OF THE DRAWINGS

FIG. 1. Kaplan-Meier analyses of overall survival of cancer patients according to the presence of antibody response to individual antigens. Detection of antibody response to antigens listed above each graph was measured in ovarian cancer patients. Associations of autoantibody responses with clinical outcome were assessed by comparing differences between curves with the log-rank method and judged significant if P<0.05. The accession numbers of transcripts coding for these antigens are listed in Table 3.

FIG. 2. Kaplan-Meier analyses of overall survival of cancer patients according to the presence of antibody response to a set of antigens. Detection of antibody response to any of the antigens listed above each graph was measured in ovarian cancer patients. Significant associations of autoantibody responses with better (A) worse (B) clinical outcome were found by comparing differences between curves with the log-rank method. The accession numbers of transcripts coding for these antigens are listed in Table 3.

### DETAILED DESCRIPTION

Cancer is a class of diseases in which a group of cells (tumor cells) display uncontrolled growth, invasion, and sometimes metastasis. Cancer antigens (also referred to herein as tumor antigens) are substances produced in tumor cells that trigger an immune response (cellular and/or humoral) in a host. Cancer antigens are useful in identifying tumor cells and are candidates for use in cancer therapy. The disclosure generally relates to methods, compositions and kits useful for the diagnosis and treatment of cancer based on newly discovered cancer antigens. In particular aspects, a panel of ovarian cancer antigens discovered in human serum samples is disclosed herein. The antigens were identified through the use of protein microarrays (polypeptide arrays) containing >8200 full-length protein antigens to detect serum antibody responses in cancer patients. Serum samples were analyzed from ovarian cancer patients as well as age-matched healthy donors. A total of 197 proteins were identified as being immunogenic (producing a humoral response) in ovarian cancer patients (see Table 3).

As used herein, the term “cancer antigens” refers to polypeptides that elicit specific immune responses (e.g., by antibodies and/or T lymphocytes) to the polypeptide when expressed by a tumor cell in a subject. In part, the disclosure relates to cancer antigens as well as the nucleic acid molecules (e.g., genes and gene transcripts) that encode them and antibodies and antigen-binding fragments that specifically bind them. The cancer antigens of the disclosure also encompass variants, homologues, and immunogenic fragments. Variants may result from alternative splicing or allelic variation of certain transcripts provided by reference in Table 3. In general, homologs and alleles typically will share at least 90% nucleotide identity and/or at least 95% amino acid identity to the sequences of the cancer antigen nucleic acids and polypeptides, respectively, in some instances will share at least 95% nucleotide identity and/or at least 97% amino acid identity, in other instances will share at least 97% nucleotide identity and/or at least 98% amino acid identity, in other instances will share at least 99% nucleotide identity and/or at least 99% amino acid identity, and in other instances will share at least 99.5% nucleotide identity and/or at least 99.5% amino acid identity. Homology can be calculated using various, publicly available software tools known in the art, such as those developed by NCBI (Bethesda, Md.) that are available through the internet. Exemplary tools include the BLAST system (e.g., using the default nucleic acid (Blastn) or protein (Blastp) search parameters) available from the website of the National Center for Biotechnology Information (NCBI) at the National Institutes of Health. Watson-Crick complements of the foregoing nucleic acids also are embraced by the invention.

The cancer antigen polypeptides of the disclosure are, among other things, useful for diagnosing cancer, particularly ovarian cancer. The diagnostic methods involve detecting specific immunoreactivity against cancer antigens in a subject. The immunoreactivity against the cancer antigens may be humoral or cellular, and is associated with a specific immune response to the cancer antigens in the subject.

In some embodiments, the antigen polypeptides are useful, among other things, for prognosing patient outcome in ovarian cancer. In some embodiments, patient outcome prognosis is based on the presence or absence, or on a determined level of a single antigen polypeptide, or autoantibody binding to such a polypeptide, in the subject. In some embodiments, patient outcome prognosis is based on the presence or absence, or on a determined level of a plurality of antigen polypeptides, or autoantibodies binding to such polypeptides, in the subject. Some embodiments provide prognostic signatures, combinations of polypeptide the presence or absence, or elevated or decreased level of which are indicative of good or bad patient outcome prognosis as described in more detail elsewhere herein. The term “subject” as used herein means any mammalian individual, including, e.g., humans and non-human mammals, such as primates, rodents, and dogs. Subjects specifically intended for diagnosis, prognosis, and treatment using the methods described herein are preferably humans.

Ovarian cancer is a type of cancer that forms in tissues of the ovary. Examples of ovarian cancers include, but are not limited to ovarian epithelial carcinoma and germ cell tumors.

Specific immunoreactivity in a subject may be detected using any of a number of methods known in the art. In some embodiments, the immunoreactivity is determined using a biological sample obtained from a subject (e.g., a human). Exemplary, biological samples include an isolated cell, an isolated tissue, saliva, gingival secretions, cerebrospinal fluid (spinal fluid), gastrointestinal fluid, mucus, urogenital secretions, synovial fluid, blood, serum, plasma, urine, cystic fluid, lymph fluid, ascites, pleural effusion, interstitial fluid, intracellular fluid, ocular fluids, seminal fluid, mammary secretions, vitreal fluid, and nasal secretions. However, biological samples are not so limited and other exemplary biological specimens will be readily apparent to one of ordinary skill in the art. In some embodiments, the biological sample is preferably a blood or serum sample.

In some embodiments, the diagnostic methods involve contacting a biological sample obtained from a subject (human) with a polypeptide (an isolated polypeptide) that corresponds to a cancer antigen disclosed herein (e.g., Table 3). A biological sample that is obtained from a subject, such as a subject with cancer, that has had a specific immune response to the corresponding cancer antigen will likely contain one or more antibodies and/or T-lymphocytes that specifically bind the polypeptide. In one embodiment, contacting the biological sample obtained from the subject with the polypeptide results in specific binding of an antibody in the biological sample with the polypeptide. This binding interaction between the antibody and polypeptide is detected for diagnostic purposes using methods known to the skilled artisan. In another embodiment, contacting the biological sample obtained from the subject with the polypeptide results in specific binding of a T-lymphocyte in the biological sample with the polypeptide. This binding interaction between the T-lymphocyte and polypeptide is detected for diagnostic purposes using methods known to the skilled artisan.

Binding between antibodies and cancer antigen polypeptides can be detected using any one of a number of art-known methods. The antigens and/or polypeptides can be in a liquid phase or bound to a solid phase carrier for detecting binding. In some embodiments, the polypeptides are immobilized on a solid surface (also referred to as a solid support), such as the polypeptide arrays used in the examples below. A variety of different solid supports are suitable, such as, beads, carriers, membrane, columns, and proteomics array. In a preferred embodiment, the solid support is the surface of a reaction chamber of a plate well. Typically, the plate well is in a multi-well plate having a number of wells selected from: 6, 12, 24, 96, 384, and 1536, but it is not so limited. For example, nano-titer plates could also be used. Examples of well known solid support materials include glass, polystyrene, polyvinyl chloride, polyvinylidene difluoride, polypropylene, polyethylene, polycarbonate, dextran, nylon, amyloses, natural and modified celluloses, such as nitrocellulose, polyacrylamides, agaroses, and magnetite. The nature of the support can be either fixed (e.g., array) or suspended in a solution (e.g., beads).

In some embodiments, polypeptide arrays are constructed by immobilizing large numbers of isolated (purified) proteins to solid support. Methods for producing polypeptide arrays are well known in the art. The methods typically involve production of proteins from an expression library, cloned into E. coli, yeast or similar system from which the expressed proteins are then purified, for example via His or GST tag affinity purification. Cell free protein transcription/translation is an alternative for synthesis of proteins which do not express well in bacterial or other in vivo systems. The purified (isolated) proteins are immobilized on the array surface (solid support surface) using art known methods. For example, proteins can be immobilized by adsorption, covalent (e.g., aldehydes) and non-covalent (e.g., biotin-streptavidin) interactions. Other methods of conjugation will be readily apparent to one of ordinary skill in the art.

The polypeptide arrays may be used to assay immunoreactivity in biological samples (e.g., for diagnostic purposes) against multiple cancer antigens in parallel. In some embodiments, the arrays are used to assay multiple antibody-antigen (polypeptide) binding interactions in a parallel. In some embodiments, immunoreactivity against up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, or more cancer antigens are tested in parallel. For ovarian cancer, the cancer antigens are selected from Table 3.

Other assays known in the art can be utilized to detect binding between antibodies and/or T-lymphocytes and cancer antigen polypeptides, for example Enzyme Linked Immunoassay (ELISA), radioimmunoassay (RIA), sandwich immunometric assay, flow cytometry, the western blot assay, immunoprecipitation assays, immunohistochemistry, immunomicroscopy, lateral flow immuno-chromatographic assays, and BIACORE® technology. Other suitable assays will be apparent to the skilled artisan.

In some embodiments, the diagnostic methods of the disclosure are useful for diagnosing cancer, particularly ovarian cancer. The methods involve contacting a biological sample obtained from a human subject with a (at least one) cancer antigen polypeptide and determining an amount of specific binding between the (at least one) cancer antigen and at least one antibody in the biological sample. The amount of specific binding is diagnostic for cancer (e.g., ovarian) in the human.

Specific binding between cancer antigens and antibodies is detected using art-known methods. In some embodiments, detectably labeled secondary antibodies (or antigen-binding fragments) are used that bind specifically to the antibodies (primary antibodies) of the biological sample. The antibodies may or may not already be bound to cancer antigens. In many cases, detectably labeled antibodies or antigen-binding fragments are commercially available (e.g., Invitrogen, by Life Technologies, Inc, Abcam, R&D Systems, etc.). In some cases, primary antibodies may be conjugated directly to a detectable label. Methods for conjugating detectable labels to antibodies or antigen-binding fragment to produce a detectably labeled antibody or antigen-binding fragments are well known in the art. Exemplary, detectable labels include radioisotopes, fluorophores, luminescent molecules, enzymes, biotin-moieties, epitope tags, various dye molecules, and molecules capable of activating chemiluminescent substrates. Exemplary enzyme labels include phosphatases (such as, Alkaline Phosphatase (ALP)) and peroxidases (such as, Horseradish Peroxidase (HRP)). Exemplary fluorophore labels include FITC, TRITC, Cy3, Cy5, Alexa Fluorescent Dyes, and Quantum Dots. Examples of other detectable labels are disclosed herein and still others will be apparent to one of ordinary skill in the art.

The selection of an appropriate detectable label depends on the nature of the assay. For example, immunoblots, dot blots, or enzyme-linked immunoassays (ELISAs) may employ enzymatic labels (e.g., HRP, ALP, etc.), that can be used to activate chemiluminescent or fluorescent substrates. Whereas binding interactions in polypeptide arrays are suitably detected using fluorophore-based labels.

In some aspects, the diagnostic methods include comparing the level (amount) of specific immunoreactivity (e.g. the level of an antibody specifically binding a certain antigen) in a biological sample to one or more reference standards. A reference standard, in some embodiments, is a set of one or more control levels (e.g., a predetermined value or range of values). The reference standard may be, for example, the level of specific immunoreactivity in a subject that does (positive control) or does not (negative control) have a cancer, e.g. an ovarian cancer. For the ovarian cancer antigens of Table 3, the reference standard may be, for example, the level of specific immunoreactivity in a subject that does or does not have a ovarian cancer. In some embodiment, the amount of immunoreactivity is compared to a background level. The skilled artisan will appreciate that the background level may be the level of immunoreactivity detected, for example, in a sample from a cancer-free subject, or in a blank sample (e.g., water, saline, etc.). In some cases, the reference standard may be a series of levels (e.g., serial dilutions) associated with varying amounts of immunoreactivity in a sample and useful for quantifying immunoreactivity (e.g., establishing an antibody titer). In some embodiments, a control or reference level is a level representing an average level in a population, for example, an average level of antibody against a specific antibody found in a random population of subjects, an age-matched population, a sex-matched population, a population of subjects not having ovarian cancer, a population of subjects having ovarian cancer, or a population meeting multiple of the foregoing criteria. These examples are not intended to be limiting and other suitable reference standards will be readily apparent to one of ordinary skill in the art.

As used herein the term “control” includes positive and negative controls which may be predetermined values that can take a variety of forms, and also may be samples of materials (e.g., biological samples) tested in parallel with the experimental materials. Examples include biological samples from control populations or control samples generated through manufacture to be tested in parallel with the experimental samples. The control(s) can be a single cut-off value, such as a median or mean, or can be established based upon related groups, such as individuals predisposed to cancer, individuals having early or late stage cancer, or individuals undergoing cancer therapy. Other examples are samples male and/or female subjects. The predetermined value of a control will depend upon the particular population selected. For example, an apparently healthy population will have a different “normal” level range than will a population which is known to have a predisposition for cancer. Accordingly, the predetermined values selected may take into account the category in which an individual falls. Appropriate ranges and categories can be selected with no more than routine experimentation by those of ordinary skill in the art. Typically the control will be based on apparently healthy individuals in an appropriate age bracket.

In some aspects, diagnostic methods are based on the detection of elevated levels of immunoreactivity against a cancer antigen that are statistically significantly different from a negative control or background levels. For example, levels of immunoreactivity against a cancer antigen that are not statistically significantly different than levels in a positive control sample obtained from a subject having cancer would be diagnostic of cancer in the subject from which the test biological sample was taken. Similarly, levels of immunoreactivity against a cancer antigen that are statistically significantly higher than immunoreactivity levels in a negative control sample obtained from a cancer-free subject would be diagnostic of cancer in the subject from which the test biological sample was taken. In contrast, levels of immunoreactivity against a cancer antigen that are not statistically significantly different than levels in a negative control sample obtained from a cancer-free subject would be indicative of a lack of a particular cancer in the subject from which the test biological sample was taken.

The level of immunoreactivity above a negative control or background that is diagnostic of cancer will vary depending on a variety of factors, including the particular cancer antigen(s) being tested and the type of cancer. In some embodiments, a level of immunoreactivity is at least about 5%, 10%, 20%, 30%, 40%, 50%, 60%, 70%, 80%, 90%, 100%, 150%, 200%, 300%, 400%, or 500% more than the level of immunoreactivity in the negative control or background sample.

Some embodiments provide methods, biomarkers, and/or biomarker signatures for the prognosis of disease course and patient outcome in ovarian cancer. Since ovarian cancer can be a terminal disease, an accurate prognosis, for example, of average life expectancy, is of great benefit for affected individuals. Some embodiments provide methods, biomarkers, and/or biomarker signatures for a determination of prognostic parameters, for example, but not limited to, projected overall survival time, or life expectancy, of an ovarian cancer patient. In some embodiments, a prognosis is formulated based on a diagnostic method described herein.

In some embodiments, a diagnostic and/or prognostic biomarker is an ovarian cancer antigen listed in Table 3. In some embodiments, a diagnostic and/or prognostic biomarker is an autoantibody specifically binding an ovarian cancer antigen listed in Table 3. In some embodiments, the presence or absence of expression of a biomarker as described herein in a subject having or being at risk of developing ovarian cancer is indicative of a higher-than-average or a lower-than-average projected life expectancy of that subject, for example, as compared to the average life expectancy of ovarian cancer patients, to the average life expectancy of age-matched ovarian cancer patients, or to the average life expectancy of ovarian cancer patients. In some embodiments, the presence of a biomarker as described herein in a subject having or being at risk of developing ovarian cancer is indicative of a higher-than-average or a lower-than-average projected life expectancy of that subject as compared to the average life expectancy of ovarian cancer patients in which the autoantibody is absent. In some embodiments, the absence of a biomarker as described herein in a subject having or being at risk of developing ovarian cancer is indicative of a higher-than-average or a lower-than-average projected life expectancy of that subject as compared to the average life expectancy of ovarian cancer patients in which the autoantibody is present.

In some embodiments, an elevated or a decreased level of a biomarker as described herein as compared to a reference or control level, for example, a level representing an average level in subjects not having and/or not being at risk of developing ovarian cancer in a subject having or being at risk of developing ovarian cancer is indicative of a higher-than-average or a lower-than-average projected life expectancy of that subject.

In some embodiments, a useful prognostic or diagnostic biomarker is an ovarian cancer antigen as described in Table 3. In some embodiments, a useful prognostic or diagnostic biomarker is an autoantibody specifically binding an ovarian cancer antigen described in Table 3. In some embodiments, the biomarker is used to prognose, for example, projected overall survival time of the subject.

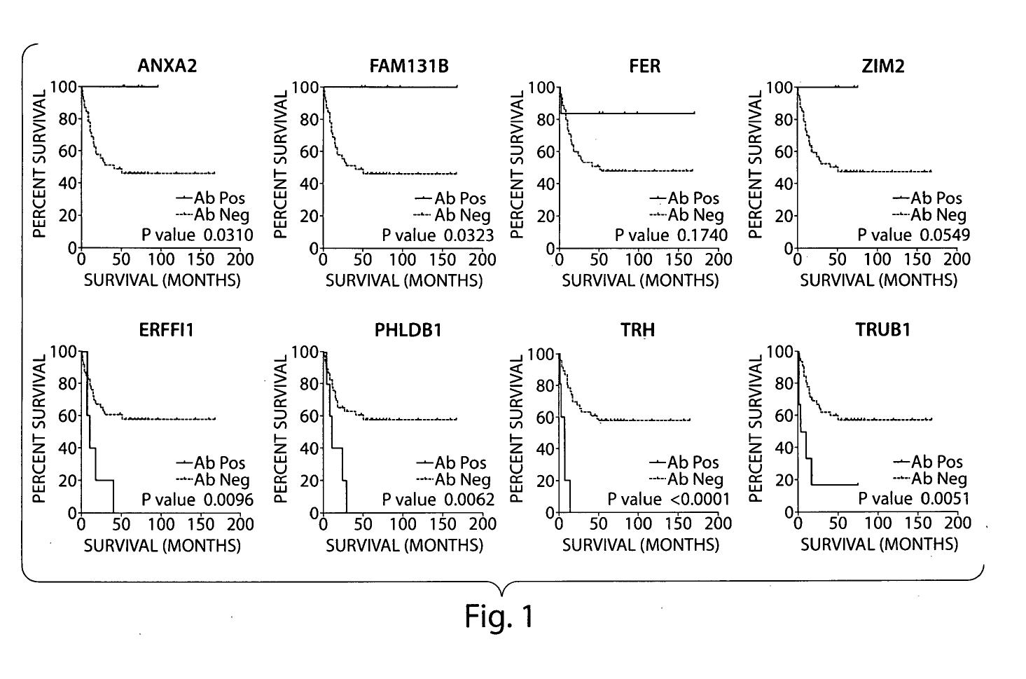

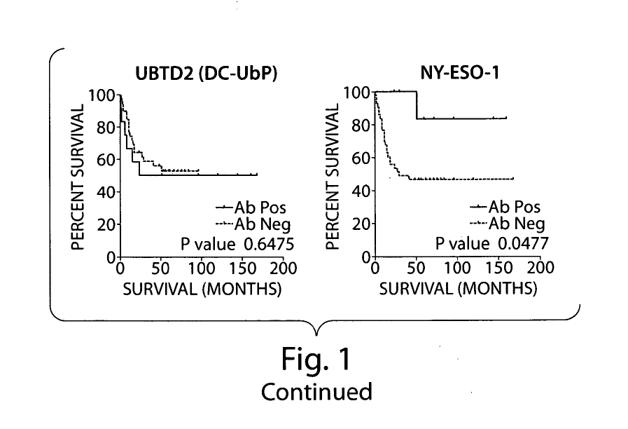

In some embodiments, the presence or an elevated level of a single autoantibody specifically binding a peptide as listed in Table 3 indicates a better-than-average or a worse-than-average prognosis (see FIG. 1). In some embodiments, the presence or an elevated level of an autoantibody specifically binding an ANXA2, FAM131B, FER, ZIM2, and/or NY-ESO-1 peptide in a subject having or being at risk of developing ovarian cancer is indicative of an increased expected survival time of the patient as compared to the average survival time of ovarian cancer patients. In some embodiments, the presence or an elevated level of an autoantibody specifically binding an ERFI1, PHLDB1, TRH, TRUB1, and/or UBTD2 (DC-UbP) peptide in a subject having or being at risk of developing ovarian cancer is indicative of a decreased expected survival time of the patient as compared to the average survival time of ovarian cancer patients.

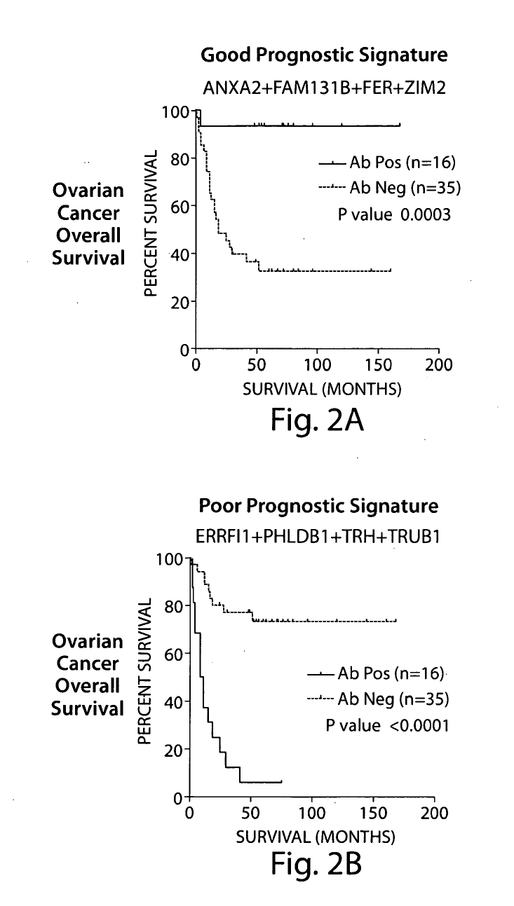

In some embodiments, the presence or elevated levels of a plurality of autoantibodies specifically binding a plurality of peptides as listed in Table 3 indicates a better-than-average or a worse-than-average prognosis (see FIG. 2). For example, in some embodiments, the presence or an elevated level of autoantibodies specifically binding ANXA2, FAM131B, FER, and ZIM2 peptides in a subject having or being at risk of developing ovarian cancer is indicative of an increased expected survival time of the patient as compared to the average survival time of ovarian cancer patients. In some embodiments, the presence or elevated levels of autoantibodies specifically binding ERFI1, PHLDB1, TRH, and TRUB1 peptides in a subject having or being at risk of developing ovarian cancer is indicative of a decreased expected survival time of the patient as compared to the average survival time of ovarian cancer patients.

The disclosure, in some aspects, also provides kits for diagnosing cancer, particularly ovarian cancer in a human. The kits can take on a variety of forms. Typically, the kits will include one or more containers housing reagents suitable for determining levels of immunoreactivity against one or more (e.g., at least two) of the polypeptides encoded by the transcripts disclosed in Table 3 in a biological sample. For example, a kit useful for diagnosing ovarian cancer may include a container housing at least two polypeptides encoded by at least two transcripts disclosed in Table 3. In some cases, the foregoing container houses immunogenic fragments of the polypeptides. The polypeptides (and/or immunogenic fragments) may be provided in a variety of formats. For example, the at least two polypeptides may be fixed to a solid substrate. The solid substrate may be a plate well. The plate well may be in a multi-well plate such as one having a number of wells selected from: 6, 12, 24, 96, 384, and 1536. In other cases, the solid substrate may be a polypeptide array surface. Optionally, the kits may contain, one or more containers housing control samples (e.g., positive and/or negative control biological samples). Also, the kits, in some cases, will include written instructions for use of the polypeptides in the diagnosis of ovarian cancer. The kits may include a reference standard of predetermined levels (e.g., control levels) of specific immunoreactivity, wherein a comparison between the levels of specific immunoreactivity in a biological sample and a reference or control is diagnostic of cancer. However, the kits are not so limited and other variations with will apparent to one of ordinary skill in the art.

In some aspects, the disclosure provides methods for treating cancer, particularly ovarian cancers. In some embodiments, the treatment methods involve administering one or more polypeptides or immunogenic fragments thereof that are encoded by the transcripts of Table 3. In other embodiments, the treatment methods involve administering antibodies or antigen binding fragments that specifically bind the cancer antigens that are encoded by the transcripts of Table 3. Treatment methods based on the cancer antigens of Table 3 are useful for treating patients having, or at risk having, ovarian cancer.

The disclosure involves the use of various materials disclosed herein to “immunize” subjects or as “vaccines”. As used herein, “immunization” or “vaccination” means increasing or activating an immune response against a cancer antigen. It does not require elimination or eradication of a cancer, or elimination of tumor cell that expresses a cancer antigen, but rather contemplates the clinically favorable enhancement of an immune response toward a cancer antigen. Methods for immunization, including formulation of a vaccine composition and selection of doses, route of administration and the schedule of administration (e.g. primary and one or more booster doses), are well known in the art. In some cases, an evaluation of vaccine compositions can be performed in humans, where the end point is to test for the presence of enhanced levels of circulating CTLs against cells bearing the antigen, to test for levels of circulating antibodies against the antigen, to test for the presence of cells expressing the antigen and so forth.

The cancer antigen polypeptides or immunogenic fragments thereof that are encoded by the transcripts of Table 3 may be administered by any one of a number of methods known in the art. In some embodiments, the polypeptides or immunogenic fragments are administered directly. As part of the immunization compositions, one or more cancer antigen polypeptides or immunogenic fragments thereof are typically administered with one or more adjuvants to induce an immune response or to increase an immune response. An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillja saponaria extract; DQS21, described in PCT application WO96/33739 (SmithKline Beecham); QS-7, QS-17, QS-18, and QS-L1 (So et al., Mol. Cells 7:178-186, 1997); incomplete Freund's adjuvant; complete Freund's adjuvant; montanide; alum; CpG oligonucleotides (see e.g. Kreig et al., Nature 374:546-9, 1995); and various water-in-oil emulsions prepared from biodegradable oils such as squalene and/or tocopherol. The antigens may be administered mixed with a combination of DQS21/MPL. Other adjuvants are known in the art and can be used in the invention (see, e.g. Goding, Monoclonal Antibodies: Principles and Practice, 2nd Ed., 1986). Methods for the preparation of mixtures or emulsions of polypeptides and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268: 1432-1434, 1995), GM-CSF and IL-18. Thus cytokines can be administered in conjunction with antigens and adjuvants to increase the immune response to the antigens.

In other embodiments, cancer antigen polypeptides or immunogenic fragments thereof that are encoded by the transcripts of Table 3 are administered by way of an plasmid vectors that express the polypeptides or immunogenic fragments. Thus, it will also be recognized that the disclosure embraces the use of expression vectors containing nucleic acids that encode cancer antigen polypeptides and immunogenic fragments thereof. Expression vectors may be administered by various art known methods. For example, expression vectors can be administered directly by intramuscular or intradermal injection, gene gun, jet injection, or liposome-mediated delivery. In vivo nucleic acid delivery using targeted liposomes also is contemplated according to the invention.

In some embodiments, expression plasmid vectors are delivered via virus vectors. Virus vectors for delivering a expression vectors encoding cancer antigen polypeptides or immunogenic fragments thereof are selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venezuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol. 26:1951-1959, 1996). A preferred virus vector is an adenovirus.

It will be understood that the foregoing virus vectors: (1) contain exogenous genetic material that can be transcribed and translated in a mammalian cell and that the expression product (polypeptide or fragment thereof) can induce an specific immune response in a host subject, and (2) contain on a surface a ligand that selectively binds to a receptor on the surface of a target cell, such as a mammalian cell, and thereby gains entry to the target cell.

In another embodiment, the delivery of nucleic acid is accomplished by ex vivo methods, i.e. by removing a cell from a subject, genetically engineering the cell to include a cancer antigen polypeptide or immunogenic fragment thereof, and reintroducing the engineered cell into the subject. One example of such a procedure is outlined in U.S. Pat. No. 5,399,346. In general, the method involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art.

As will be apparent to the skilled artisan, some therapeutic approaches based upon the disclosure are premised on a response by a subject's immune system, leading to lysis of antigen presenting cells, such as cancer cells which present one or more cancer antigens of the invention. One such approach is the administration of autologous CTLs specific to a cancer antigen/MHC complex to a subject with abnormal cells of the phenotype at issue. It is within the ability of one of ordinary skill in the art to develop such CTLs in vitro. An example of a method for T cell differentiation is presented in International application number PCT/US96/05607. Generally, a sample of cells taken from a subject, such as blood cells, are contacted with a cell presenting the complex and capable of provoking CTLs to proliferate. The target cell can be a transfectant, such as a COS cell. These transfectants present the desired complex of their surface and, when combined with a CTL of interest, stimulate its proliferation. COS cells are widely available, as are other suitable host cells. Specific production of CTL clones is well known in the art. The clonally expanded autologous CTLs then are administered to the subject.

Another method for selecting antigen-specific CTL clones has recently been described (Altman et al., Science 274:94-96, 1996; Dunbar et al., Curr. Biol. 8:413-416, 1998), in which fluorogenic tetramers of MHC class I molecule/peptide complexes are used to detect specific CTL clones. Briefly, soluble MI-IC class I molecules are folded in vitro in the presence of β₂-microglobulin and a peptide antigen which binds the class I molecule. After purification, the MHC/peptide complex is purified and labeled with biotin. Tetramers are formed by mixing the biotinylated peptide-MHC complex with labeled avidin (e.g. phycoerythrin) at a molar ratio or 4:1. Tetramers are then contacted with a source of CTLs such as peripheral blood or lymph node. The tetramers bind CTLs which recognize the peptide antigen/MHC class I complex. Cells bound by the tetramers can be sorted by fluorescence activated cell sorting to isolate the reactive CTLs. The isolated CTLs then can be expanded in vitro for use as described herein.

To detail a therapeutic methodology, referred to as adoptive transfer (Greenberg, J. Immunol. 136(5): 1917, 1986; Riddel et al., Science 257: 238, 1992; Lynch et al, Eur. J. Immunol. 21: 1403-1410,1991; Kast et al., Cell 59: 603-614, 1989), cells presenting the desired complex (e.g., dendritic cells) are combined with CTLs leading to proliferation of the CTLs specific thereto. The proliferated CTLs are then administered to a subject with a tumor cell presenting the particular complex. The CTLs then lyse the tumor cells, thereby achieving the desired therapeutic goal.

The foregoing therapy assumes that at least some of the subject's tumor cells present the relevant HLA/cancer associated antigen complex. This can be determined very easily, as the art is very familiar with methods for identifying cells which present a particular HLA molecule, as well as how to identify cells expressing DNA of the pertinent sequences, in this case a cancer antigen sequence. Once cells presenting the relevant complex are identified via the foregoing screening methodology, they can be combined with a sample from a patient, where the sample contains CTLs. If the complex presenting cells are lysed by the mixed CTL sample, then it can be assumed that a cancer antigen is being presented, and the subject is an appropriate candidate for the therapeutic approaches set forth herein.

Adoptive transfer is not the only form of therapy that is available in accordance with the disclosure. CTLs can also be provoked in vivo, using a number of approaches. One approach is the use of non-proliferative cells expressing the complex. The cells used in this approach may be those that normally express the complex, such as irradiated tumor cells or cells transfected with one or both of the genes necessary for presentation of the complex (i.e. the antigenic peptide and the presenting MHC molecule). Chen et al. (Proc. Natl. Acad. Sci. USA 88: 110-114,1991) exemplifies this approach, showing the use of transfected cells expressing HPV E7 peptides in a therapeutic regime. Various cell types may be used. Similarly, vectors carrying one or more cancer antigen encoding nucleic acids may be used. Viral or bacterial vectors are especially preferred. For example, nucleic acids which encode a cancer antigen polypeptide may be operably linked to promoter and enhancer sequences which direct expression of the cancer antigen polypeptide in certain tissues or cell types. The nucleic acid may be incorporated into an expression vector.

The disclosure also involves the use of binding agents, such as antibodies and antigen-binding fragments, that bind to cancer antigens. Such agents can be used in methods of the invention including the diagnosis and/or treatment of cancer. Such agents also may be used to inhibit the native activity of the cancer antigen polypeptides, for example, by binding to such polypeptides in vivo.

The binding agents of the disclosure bind to a cancer antigen polypeptide, including immunogenic fragments thereof that are encoded by the transcripts disclosed in Table 3. In preferred embodiments, the binding agent is an antibody or antibody fragment, more preferably, an Fab or F(ab)2 fragment of an antibody or a single domain antibody. Typically, the fragment includes a CDR3 region that is selective for a cancer antigen of the disclosure. Any of the various types of antibodies can be used for this purpose, including polyclonal antibodies, monoclonal antibodies, humanized antibodies, chimeric antibodies and single domain antibodies.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762, and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present disclosure also provides for F(ab′)2, Fab, Fv, and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present disclosure also includes so-called single chain antibodies.

The disclosure also embraces nucleic acids encoding the foregoing binding agents that bind to the cancer antigen polypeptides and immunogenic fragments thereof that are encoded by the transcripts disclosed in Table 3. Nucleic acids encoding the foregoing binding agents may be administered using any of the expression vector and virus vector delivery methods disclosed herein and known in the art.

Thus, the disclosure provides agents which bind to cancer antigens encoded by the transcripts of Table 3, and optionally immunoreactive fragments of the cancer antigen polypeptides. Such binding agents have a variety of uses. For example the agents may be useful as a passive immunization agents against cancer, particularly ovarian cancer. In addition, such binding agents can be used to selectively target drugs, toxins or other molecules (including detectable labels for diagnostic purposes) to cells which express the cancer antigens. In this manner, for example, cells present in tumors which express cancer antigen can be treated with cytotoxic compounds that are selective for the cancer antigens. Such binding agents also can be used to inhibit the native activity of the cancer antigen, for example, to further characterize the functions of these molecules.

The antibodies or antigen binding fragments, of the present invention can also be used to therapeutically target cells expressing cancer antigens. In a preferred embodiment, antibodies can be used to target antigens expressed on the cell surface. These antibodies can be linked not only to a detectable marker but also an antitumor agent or an immunomodulator. Antitumor agents can include cytotoxic agents, such as cytotoxic radionuclides or radiotherapeutic isotopes preferably is an alpha-emitting isotope such as ²²⁵Ac, ²¹¹At, ²¹²Bi, ²¹³Bi, ²¹²Pb, ²²⁴Ra or ²²³Ra. Alternatively, the cytotoxic radionuclide may a beta-emitting isotope such as ¹⁸⁶Rh, ¹⁸⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes ¹²⁵I, ¹²³I or ⁷⁷Br.

Other suitable antitumor agents include, but are not limited to, the following compounds: Antineoplastic agents such as: Acivicin; Aclarubicin; Acodazole Hydrochloride; Acronine; Adozelesin; Adriamycin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Buniodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorombucil; Cirolemycin; Cisplatin; Cladribine; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Dexormaplatin; Dezaguanine; Dezaguanine Ifesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Fluorocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-1a; Interferon Gamma-1b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin, Mitosper; Mitotane; Mitoxantrone Hydrochloride; Mycophenolic Acid; Nocodazole; Nogalamycin; Ormaplatin; Oxisuran; Paclitaxel Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP-53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate, Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′-Deoxyformycin; 9-aminocamptothecin; raltitrexed; N-propargyl-5,8-dideazafolic acid, 2-chloro-2′-arabino-fluoro-2′-deoxyadenosine; 2-chloro-2′-deoxyadenosine; anisomycin; trichostatin A; hPRL-G129R; CEP-751; linomide; Piritrexim Isethionate; Sitogluside; Tamsulosin Hydrochloride and Pentomone.

Other toxins include poisonous lectins, plant toxins such as ricin, abrin, modeccin, botulina and diphtheria toxins. Combinations of the various toxins could also be coupled to an antibody molecule thereby accommodating variable cytotoxicity. Other chemotherapeutic agents are known to those skilled in the art.

The coupling of one or more antitumor agents to the antibody is envisioned to include many chemical mechanisms, for instance covalent binding, affinity binding, intercalation, coordinate binding, and complexation. The toxic compounds used to prepare the immunotoxins are attached to the antibodies or antigen-binding fragments thereof by standard protocols known in the art.

In certain embodiments, the disclosure provides nucleic acids, which include additions, substitutions and deletions of one or more nucleotides, that encode cancer antigens. The cancer antigen polypeptides encoded by these nucleic acids substantially retain the immunogenic properties (specific immune response by antibodies and/or T lymphocytes) of the wild-type polypeptide. In certain embodiments, the modified polypeptides are preferably polypeptides having conservative amino acid substitutions as are well known in the art.

According to yet another aspect of the invention, an expression vector comprising any of the cancer antigen encoding nucleic acid molecules of the invention (e.g., transcripts disclosed in Table 3, and variants and homologues thereof), preferably operably linked to a promoter is provided. In a related aspect, host cells transformed or transfected with such expression vectors also are provided. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, cloning vectors and virus genomes. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., β-galactosidase or alkaline phosphatase, and genes which visibly affect the phenotype of transformed or transfected cells, hosts, colonies or plaques, e.g., green fluorescent protein. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

It will also be recognized that the invention embraces the use of the cancer antigen encoding nucleic acid molecules in expression vectors. Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 2000. Cells are genetically engineered by the introduction into the cells of an expression vector encoding a cancer antigen, fragments, or variants thereof. The host cell may be of a wide variety of tissue types, including mast cells, fibroblasts, oocytes, and lymphocytes, and may be primary cells and cell lines. Specific examples include dendritic cells, peripheral blood leukocytes, bone marrow stem cells and embryonic stem cells. The expression vectors require that the pertinent sequence, i.e., those nucleic acids described herein, be operably linked to a promoter.

Preferred systems for mRNA expression in mammalian cells are those such as pcDNA (Invitrogen) that contain a selectable marker (which facilitates the selection of stably transfected cell lines) and contain the human cytomegalovirus (CMV) enhancer-promoter sequences. Gateway™ vectors such as pCMVSport-6 vectors from Invitrogen are particularly suitable for rapid cloning. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element (See for example, Nakayama et al. (2005). Journal of Virology, 79:8870-8877). Another expression vector is the pEF-BOS plasmid containing the promoter of polypeptide Elongation Factor 1, which stimulates efficiently transcription in vitro. The plasmid is described by Mizushima and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is described by Warnier et al., in intradermal injection in mice for immunization against P1A (Int. J. Cancer, 67:303-310, 1996). Other examples include Ad5 based Adenoviral expression vectors such as those described in Catalucci D, et al., J. Virol. 2005 May; 79(10):6400-9.

The disclosure, in another aspect, provides isolated polypeptides (including whole proteins and partial proteins) of the cancer antigens. Immunogenic fragments of the polypeptides that bind antibodies or MHC molecules are also provided. One important activity of the polypeptides and immunogenic fragments is the ability to provoke in a subject an immune response. As will be recognized by those skilled in the art, the size of the fragment that can be used for inducing an immune response will depend upon factors such as whether the epitope recognized by an antibody is a linear epitope or a conformational epitope or the particular MHC molecule that binds to and presents the fragment (e.g. HLA class I or II). Thus, some immunogenic fragments of cancer antigen polypeptides will consist of longer segments while others will consist of shorter segments, (e.g. 5, 6, 7, 8, 9, 10, 11 or 12 or more amino acids long, including each integer up to the full length of the cancer antigen polypeptide). Those skilled in the art are well versed in methods for selecting immunogenic fragments of polypeptides.

The disclosure also embraces variants of the cancer antigen polypeptides described herein. As used herein, a “variant” of a cancer antigen polypeptide is a polypeptide which contains one or more modifications to the primary amino acid sequence of a cancer antigen polypeptide. Modifications which create a cancer antigen variant can be made to 1) to reduce or eliminate an activity; 2) to enhance a property, such as in vivo protein stability; 3) to provide a novel activity or property, such as addition of an antigenic epitope or addition of a detectable moiety; or 4) to provide equivalent or better binding to a MHC molecule.

Modifications to a cancer antigen polypeptide are typically made to the nucleic acid which encodes the polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the cancer antigen amino acid sequence. One of skill in the art will be familiar with methods for predicting the effect on protein conformation of a change in protein sequence, and can thus “design” a variant according to known methods. One example of such a method is described by Dahiyat and Mayo in Science 278:82 87, 1997, whereby proteins can be designed de novo. The method can be applied to a known protein to vary a only a portion of the polypeptide sequence. By applying the computational methods of Dahiyat and Mayo, specific variants can be proposed and tested to determine whether the variant retains a desired conformation.

The skilled artisan will also realize that conservative amino acid substitutions may be made in cancer antigen polypeptides to provide functionally equivalent variants of the foregoing polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 2000, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the cancer antigens disclosed herein and retain the specific antibody-binding characteristics of the antigens.

Likewise, upon determining that a peptide derived from a cancer antigen polypeptide is presented by an MHC molecule and recognized by antibodies or T lymphocytes (e.g., helper T cells or CTLs), one can make conservative amino acid substitutions to the amino acid sequence of the peptide, particularly at residues which are thought not to be direct contact points with the MHC molecule. For example, methods for identifying functional variants of HLA class II binding peptides are provided in a published PCT application of Strominger and Wucherpfennig (PCT/US96/03182). Peptides bearing one or more amino acid substitutions also can be tested for concordance with known HLA/MHC motifs prior to synthesis using, e.g. the computer program described by D'Amaro and Drijfhout (D'Amaro et al., Human Immunol. 43:13-18, 1995; Drijfhout et al., Human Immunol. 43:1-12, 1995). The substituted peptides can then be tested for binding to the MHC molecule and recognition by antibodies or T lymphocytes when bound to MHC. These variants can be tested for improved stability and are useful, inter alia, in vaccine compositions.

Conservative amino-acid substitutions in the amino acid sequence of cancer antigen polypeptides to produce functionally equivalent variants typically are made by alteration of a nucleic acid encoding a cancer antigen polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene, or fragment, encoding a cancer antigen polypeptide, or fragment. Where amino acid substitutions are made to a small unique fragment of a polypeptide, such as an antigenic epitope recognized by autologous or allogeneic sera or T lymphocytes, the substitutions can be made by directly synthesizing the peptide. The activity of functionally equivalent variants of cancer antigen polypeptides can be tested by cloning the gene encoding the altered cancer antigen polypeptide into a bacterial or mammalian expression vector, introducing the vector into an appropriate host cell, expressing the altered polypeptide, and testing for a functional capability of the cancer antigen polypeptides as disclosed herein. Peptides that are chemically synthesized can be tested directly for function, e.g., for binding to antisera recognizing associated antigens.

According to a further aspect of the disclosure, compositions containing the nucleic acid molecules, polypeptides and immunogenic fragments thereof, and binding agents of the invention are provided. The compositions contain any of the foregoing therapeutic agents in an optional pharmaceutically acceptable carrier. Thus, in a related aspect, the invention provides a method for forming a medicament that involves placing a therapeutically effective amount of the therapeutic agent in the pharmaceutically acceptable carrier to form one or more doses. The effectiveness of treatment or prevention methods of the invention can be determined using the diagnostic methods described herein.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The therapeutics of the invention can be administered by any conventional route, including injection or by gradual infusion over time. The administration may, for example, be oral, intravenous, intratumoral, intraperitoneal, intramuscular, intracavity, subcutaneous, or transdermal.

The compositions of the invention are administered in effective amounts. An “effective amount” is that amount of an active agent (e.g., a cancer antigen polypeptide, nucleic acid encoding cancer antigen polypeptide, or antibody that binds a cancer antigen polypeptide) in a composition that alone, or together with further doses, produces the desired response, e.g. increases an immune response to the cancer antigen polypeptide. In the case of treating a particular disease or condition characterized by immunoreactivity against one or more cancer antigen polypeptides, such as ovarian cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of a therapeutic agent (e.g., a cancer antigen polypeptide, nucleic acid encoding cancer antigen polypeptide, or antibody that binds a cancer antigen polypeptide) for producing the desired response in a unit of weight or volume suitable for administration to a patient. The response can, for example, be measured by determining the immune response following administration of the composition via a reporter system by measuring downstream effects such as immunoreactivity, or by measuring the physiological effects of the composition, such as regression of a tumor or decrease of disease symptoms. Other assays will be known to one of ordinary skill in the art and can be employed for measuring the level of the response.

The doses of compositions administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, treatments for eliciting or increasing an immune response are formulated and administered in doses of binding agents, polypeptides or immunogenic fragments thereof, between 1 ng and 1 mg, and preferably between 10 ng and 100 μg, according to any standard procedure in the art. Where nucleic acids encoding cancer antigen polypeptides or fragments thereof are employed, doses of between 1 ng and 0.1 mg generally will be formulated and administered according to standard procedures. Other protocols for the administration of cancer antigen-based compositions will be known to one of ordinary skill in the art, in which the dose amount, schedule of injections, sites of injections, mode of administration (e.g., intra-tumoral) and the like vary from the foregoing. Administration of compositions to mammals other than humans, e.g. for testing purposes or veterinary therapeutic purposes, is carried out under substantially the same conditions as described above.

Where cancer antigen polypeptides or immunogenic fragments thereof are used for vaccination, modes of administration that effectively deliver the polypeptide and adjuvant, such that an immune response to the polypeptide is increased, can be used. For administration of a polypeptide in adjuvant, preferred methods include intradermal, intravenous, intramuscular and subcutaneous administration. Although these are preferred embodiments, the invention is not limited by the particular modes of administration disclosed herein. Standard references in the art (e.g., Remington's Pharmaceutical Sciences, 18th edition, 1990) provide modes of administration and formulations for delivery of immunogens with adjuvant or in a non-adjuvant carrier.

The pharmaceutical compositions of the disclosure are also not limited to one cancer-antigen polypeptide or immunogenic fragment thereof. In some embodiments, the compositions comprise up to 2, 3, 4, 5, 6, 7, 8, 9, 10, 11, 12, 13, 14, 15, 16, 17, 18, 19, 20, or more different cancer antigen polypeptides or immunogenic fragments thereof that are encoded by the transcripts disclosed in Table 3. In some embodiments, the compositions comprise up to 2, 3, 4, 5, 6, 7, 8, 9, 10, 11, 12, 13, 14, 15, 16, 17, 18, 19, 20, or more different vectors that express cancer antigen polypeptides or immunogenic fragments thereof that are encoded by the transcripts disclosed in Table 3. In other embodiments, the compositions comprise up to 2, 3, 4, 5, 6, 7, 8, 9, 10, 11, 12, 13, 14, 15, 16, 17, 18, 19, 20, or more different binding agents that bind cancer antigen polypeptides that are encoded by the transcripts disclosed in Table 3.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, and lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

The pharmaceutical agents of the invention may be administered alone, in combination with each other, and/or in combination with other anti-cancer drug therapies and/or treatments. These therapies and/or treatments may include, but are not limited to: surgical intervention, chemotherapy, radiotherapy, and adjuvant systemic therapies.

The invention also provides a pharmaceutical kit comprising one or more containers comprising one or more of the pharmaceutical compounds or agents of the invention. Additional materials may be included in any or all kits of the invention, and such materials may include, but are not limited to buffers, water, enzymes, tubes, control molecules, etc. The kit may also include instructions for the use of the one or more pharmaceutical compounds or agents of the invention for the treatment of cancer.

TABLE 1
Ovarian Cancer Patients

| Sample | | | | | | |
|---|---|---|---|---|---|---|
| Number | Age | Gender | Stage | Grade | HistoType | Clinical Outcome |
| SRM02504 | 46 | F | IIIC | 3 | serous | Long-term survivors |
| SRM01856 | 74 | F | IIIC | 3 | endometroid | Long-term survivors |
| SRM02800 | 38 | F | IIIC | 3 | NA | Long-term survivors |
| SRM00033 | 42 | F | IIIC | 1 | Serous | Long-term survivors |
| SRM00053 | 51 | F | IIIC | 3 | Serous | Long-term survivors |
| SRM02752 | 41 | F | IIIC | NA | Serous | Long-term survivors |
| SRM15024 | 62 | F | IIIC | 3 | Serous | Long-term survivors |
| SRM02634 | 57 | F | IIIC | 3 | Serous | Long-term survivors |
| SRM00355 | 26 | F | IIIB | 3 | Serous | Long-term survivors |
| SRM00352 | 59 | F | IIIC | 2 | Serous | Long-term survivors |
| SRM00535 | 77 | F | IIIC | 3 | Serous | Long-term survivors |
| SRM00558 | 77 | F | IIIC | 3 | Mixed | Long-term survivors |
| SRM01489 | 48 | F | IIIC | 3 | Serous | Long-term survivors |
| SRM00859 | 58 | F | IIIC | 3 | Serous | Long-term survivors |
| SRM00842 | 34 | F | IIIA | 1 | Serous | Long-term survivors |
| SRM01527 | 66 | F | IIIC | 3 | Serous | Long-term survivors |
| SRM00959 | 66 | F | IIIC | 3 | Serous | Long-term survivors |
| SRM01859 | 46 | F | IIIC | 3 | Mixed | Long-term survivors |
| SRM02258 | 76 | F | IIIC | 3 | serous | Long-term survivors |
| SRM01467 | 44 | F | IV | 3 | serous | Long-term survivors |
| SRM00722 | 58 | F | IIIC | 3 | serous | Long-term survivors |
| SRM00393 | 81 | F | IIIC | 3 | serous | Long-term survivors |
| SRM01005 | 70 | F | IIIC | 3 | serous | Long-term survivors |
| SRM00860 | 58 | F | IIIC | 3 | serous | Long-term survivors |
| SRM00943 | 66 | F | IIIC | 3 | serous | Long-term survivors |
| SRM00473 | 53 | F | IIIC | 3 | serous | Long-term survivors |
| SRM00743 | 64 | F | IIIC | 3 | serous | Long-term survivors |
| SRM14182 | 55 | F | IIIC | 3 | mixed | Short-term survivors |
| SRM02162 | 58 | F | IIIC | 2 | serous | Short-term survivors |
| SRM15555 | 58 | F | IV | 3 | serous | Short-term survivors |
| SRM03582 | 75 | F | IV | 2 | serous | Short-term survivors |
| SRM14175 | 58 | F | IV | 3 | serous | Short-term survivors |
| SRM14085 | 71 | F | IIIC | 3 | serous | Short-term survivors |
| SRM15261 | 75 | F | IIIC | | serous | Short-term survivors |
| SRM04744 | 45 | F | IIIC | 3 | clear cell | Short-term survivors |
| SRM14326 | 66 | F | IIIC | 2 | mucinous | Short-term survivors |
| SRM02893 | 49 | F | IIIC | 3 | clear cell | Short-term survivors |
| SRM00862 | 78 | F | IIIC | 3 | serous | Short-term survivors |
| SRM04752 | 62 | F | IIIC | 3 | serous | Short-term survivors |
| SRM01518 | 48 | F | IIIC | 3 | serous | Short-term survivors |
| SRM14667 | 59 | F | IIIC | 2 | serous | Short-term survivors |
| SRM01374 | 82 | F | IIIC | 3 | serous | Short-term survivors |
| SRM00809 | 68 | F | IIIC | 3 | serous | Short-term survivors |
| SRM02393 | 81 | F | IIIB | 3 | serous | Short-term survivors |
| SRM02544 | 88 | F | IIIC | 3 | serous | Short-term survivors |
| SRM00622 | 77 | F | IIIC | 3 | serous | Intermediate-term survivors |
| SRM01798 | 62 | F | IIIC | 3 | serous | Intermediate-term survivors |
| SRM01546 | 80 | F | IIIC | 3 | serous | Intermediate-term survivors |
| SRM00312 | 64 | F | IIIC | 3 | serous | Intermediate-term survivors |
| SRM04750 | 73 | F | IIIC | 3 | serous | Intermediate-term survivors |
| SRM03694 | 79 | F | IIIC | 3 | mixed | Intermediate-term survivors |
| SRM02547 | 72 | F | IIIC | 3 | serous | Intermediate-term survivors |

TABLE 2
Healthy Donors

| Sample Number | Age | Gender |
|---|---|---|
| 001000017 | 53 | M |
| 001000018 | 92 | M |
| 001000040 | 44 | M |
| 001000058 | 38 | M |
| 001000059 | 59 | M |
| 001000135 | 46 | F |
| 001000186 | 57 | F |
| 001000242 | 79 | M |
| 001000245 | 64 | M |
| 001000258 | 75 | F |
| 001000260 | 71 | F |
| 001000279 | 58 | F |
| 001000281 | 73 | M |
| 001000303 | 58 | M |
| 001000308 | 77 | F |
| 001000310 | 58 | M |
| 001000320 | 81 | F |
| 001000325 | 51 | M |
| 001000331 | 80 | F |
| 001000333 | 81 | F |
| 001000340 | 62 | F |
| 001000341 | 66 | F |
| 001000376 | 73 | F |
| 001000387 | 48 | F |
| 001000389 | 72 | M |
| 001000392 | 62 | M |
| 001000394 | 64 | F |
| 001000398 | 78 | M |
| 001000405 | 66 | M |
| 001000424 | 75 | F |
| 001000451 | 55 | M |
| 001000466 | 38 | M |
| 001000510 | 73 | F |
| 001000651 | 38 | F |
| 001000712 | 66 | M |
| 001000713 | 42 | F |
| 001001170 | 46 | F |
| Con-1 | 61 | F |
| Con-7 | 66 | M |
| Con-11 | 50 | M |
| Con-12 | 58 | M |
| Con-20 | 64 | M |
| Con-34 | 87 | M |
| Con-35 | 50 | M |
| Con-36 | 44 | M |
| Con-38 | 60 | F |
| Con-39 | 78 | F |
| Con-40 | 43 | F |
| Con-41 | 81 | M |
| Con-46 | 71 | M |
| Con-67 | 50 | F |
| Con-68 | 62 | F |
| Con-75 | 60 | F |

TABLE 3
Ovarian Cancer Antigens

| | | | | | Score | Score | |
|---|---|---|---|---|---|---|---|
| | | | % | % | (average | (average | |
| | | | Response | Response | ratio over | ratio over | Final Score |
| | | | in | in | cutoff) for | cutoff) for | (Ovarian- |
| Locus | Name | Symbol | Healthy | Ovarian | Healthy | Ovarian | Healthy) |
| BC033010.1 | acrosin binding | ACRBP | 2% | 6% | 1.06 | 1.66 | 5% |
| | protein | | | | | | |
| NM_001626.2 | v-akt murine | AKT2 | 2% | 6% | 1.74 | 10.32 | 11% |
| | thymoma viral | | | | | | |
| | oncogene | | | | | | |
| | homolog 2 | | | | | | |
| BC035969.1 | albumin | ALB | 2% | 6% | 1.26 | 1.86 | 5% |
| NM_018466.2 | asparagine- | ALG13 | 2% | 8% | 1.85 | 2.31 | 8% |
| | linked | | | | | | |
| | glycosylation | | | | | | |
| | 13 homolog (S. cerevisiae) | | | | | | |
| NM_006401.1 | acidic (leucine- | ANP32B | 4% | 10% | 1.74 | 2.44 | 9% |
| | rich) nuclear | | | | | | |
| | phosphoprotein | | | | | | |
| | 32 family, | | | | | | |
| | member B | | | | | | |
| BC023990.1 | annexin A2 | ANXA2 | 2% | 12% | 2.72 | 1.52 | 11% |
| NM_001641.2 | APEX nuclease | APEX1 | 4% | 16% | 1.99 | 2.41 | 16% |
| | (multifunctional | | | | | | |
| | DNA repair | | | | | | |
| | enzyme) 1 | | | | | | |
| BC060828.1 | AT rich | ARID3A | 2% | 8% | 2.45 | 2.23 | 8% |
| | interactive | | | | | | |
| | domain 3A | | | | | | |
| | (BRIGHT- | | | | | | |
| | like) | | | | | | |
| NM_004311.1 | ADP- | ARL3 | 4% | 12% | 1.23 | 2.04 | 11% |
| | ribosylation | | | | | | |
| | factor-like 3 | | | | | | |
| NM_018184.1 | ADP- | ARL8B | 2% | 6% | 1.16 | 2.69 | 6% |
| | ribosylation | | | | | | |
| | factor-like 8B | | | | | | |
| NM_052936.2 | ATG4 | ATG4A | 0% | 4% | 1.00 | 5.87 | 7% |
| | autophagy | | | | | | |
| | related 4 | | | | | | |
| | homolog A (S. cerevisiae) | | | | | | |
| NM_032352.1 | breast cancer | BRMS1L | 2% | 14% | 1.95 | 1.73 | 14% |
| | metastasis- | | | | | | |
| | suppressor 1- | | | | | | |
| | like | | | | | | |
| NM_001007246.1 | bromodomain | BRWD1 | 2% | 10% | 7.05 | 2.81 | 10% |
| | and WD repeat | | | | | | |
| | domain | | | | | | |
| | containing 1 | | | | | | |
| BC050645.1 | Bystin-like | BYSL | 2% | 10% | 1.98 | 3.52 | 13% |
| NM_017907.1 | chromosome 11 | C11orf59 | 2% | 10% | 1.57 | 2.09 | 10% |
| | open reading | | | | | | |
| | frame 59 | | | | | | |
| BC016854.1 | chromosome 11 | C11orf67 | 4% | 10% | 1.60 | 5.26 | 13% |
| | open reading | | | | | | |
| | frame 67 | | | | | | |
| NM_017822.2 | chromosome 12 | C12orf41 | 4% | 12% | 5.68 | 4.16 | 12% |
| | open reading | | | | | | |
| | frame 41 | | | | | | |
| NM_033201.1 | chromosome 16 | C16orf45 | 4% | 14% | 7.89 | 6.02 | 17% |
| | open reading | | | | | | |
| | frame 45 | | | | | | |
| BC022189.2 | chromosome 17 | C17orf47 | 0% | 10% | 1.00 | 2.00 | 12% |
| | open reading | | | | | | |
| | frame 47 | | | | | | |
| NM_152266.1 | chromosome 19 | C19orf40 | 0% | 10% | 1.00 | 2.44 | 13% |
| | open reading | | | | | | |
| | frame 40 | | | | | | |
| BC052573.1 | chromosome 19 | C19orf46 | 0% | 10% | 1.00 | 2.12 | 13% |
| | open reading | | | | | | |
| | frame 46 | | | | | | |
| NM_019099.1 | chromosome 1 | C1orf183 | 0% | 6% | 1.00 | 1.74 | 7% |
| | open reading | | | | | | |
| | frame 183 | | | | | | |
| NM_022104.1 | chromosome 20 | C20orf67 | 0% | 10% | 1.00 | 6.42 | 18% |
| | open reading | | | | | | |
| | frame 67 | | | | | | |
| BC009485.1 | chromosome 4 | C4orf16 | 0% | 4% | 1.00 | 4.33 | 6% |
| | open reading | | | | | | |
| | frame 16 | | | | | | |
| NM_145063.1 | chromosome 6 | C6orf130 | 0% | 14% | 1.00 | 1.56 | 16% |
| | open reading | | | | | | |
| | frame 130 | | | | | | |
| BC022043.1 | chromosome 7 | C7orf36 | 4% | 12% | 1.57 | 3.08 | 13% |
| | open reading | | | | | | |
| | frame 36 | | | | | | |
| BC009074.1 | chromosome 8 | C8orf70 | 0% | 6% | 1.00 | 2.69 | 8% |
| | open reading | | | | | | |
| | frame 70 | | | | | | |
| NM_032596.3 | chromosome 9 | C9orf24 | 0% | 4% | 1.00 | 4.68 | 7% |
| | open reading | | | | | | |
| | frame 24 | | | | | | |
| NM_012117.1 | chromobox | CBX5 | 2% | 12% | 1.34 | 2.68 | 14% |
| | homolog 5 | | | | | | |
| | (HP1 alpha | | | | | | |
| | homolog, | | | | | | |
| | Drosophila) | | | | | | |
| NM_144982.1 | coiled-coil | CCDC131 | 2% | 8% | 1.35 | 1.48 | 7% |
| | domain | | | | | | |
| | containing 131 | | | | | | |
| NM_016360.1 | coiled-coil | CCDC44 | 4% | 16% | 1.53 | 3.51 | 19% |
| | domain | | | | | | |
| | containing 44 | | | | | | |
| NM_000626.1 | CD79b | CD79B | 0% | 6% | 1.00 | 1.86 | 7% |
| | molecule, | | | | | | |
| | immunoglobulin- | | | | | | |
| | associated | | | | | | |
| | beta | | | | | | |
| NM_003607.1 | CDC42 binding | CDC42BPA | 0% | 6% | 1.00 | 1.97 | 7% |
| | protein kinase | | | | | | |
| | alpha (DMPK- | | | | | | |
| | like) | | | | | | |
| NM_006779.2 | CDC42 effector | CDC42EP2 | 4% | 10% | 2.18 | 2.48 | 8% |
| | protein (Rho | | | | | | |
| | GTPase | | | | | | |
| | binding) 2 | | | | | | |
| BC010451.1 | CDC42 effector | CDC42EP4 | 2% | 12% | 1.27 | 1.92 | 13% |
| | protein (Rho | | | | | | |
| | GTPase | | | | | | |
| | binding) 4 | | | | | | |
| NM_145810.1 | cell division | CDCA7 | 2% | 8% | 1.42 | 4.96 | 11% |
| | cycle | | | | | | |
| | associated 7 | | | | | | |
| BC064145.1 | CDK5 | CDKAL1 | 2% | 8% | 1.11 | 10.99 | 15% |
| | regulatory | | | | | | |
| | subunit | | | | | | |
| | associated | | | | | | |
| | protein 1-like 1 | | | | | | |
| NM_018413.2 | carbohydrate | CHST11 | 0% | 4% | 1.00 | 2.67 | 5% |
| | (chondroitin 4) | | | | | | |
| | sulfotransferase | | | | | | |
| | 11 | | | | | | |
| BC070203.1 | collagen, type | COL4A6 | 0% | 4% | 1.00 | 3.76 | 6% |
| | IV, alpha 6 | | | | | | |
| NM_007263.3 | coatomer | COPE | 2% | 10% | 1.51 | 1.99 | 10% |
| | protein | | | | | | |
| | complex, | | | | | | |
| | subunit epsilon | | | | | | |
| BC015634.1 | coenzyme Q3 | COQ3 | 4% | 12% | 3.37 | 3.41 | 12% |
| | homolog, | | | | | | |
| | methyltransferase | | | | | | |
| | (S. cerevisiae) | | | | | | |
| NM_006067.3 | COX4 neighbor | COX4NB | 0% | 4% | 1.00 | 2.85 | 6% |
| NM_024695.1 | complexin 3 | CPLX3 | 4% | 8% | 1.47 | 2.56 | 6% |
| NM_130898.1 | cAMP | CREB3L4 | 2% | 8% | 1.69 | 1.96 | 8% |
| | responsive | | | | | | |
| | element | | | | | | |
| | binding protein | | | | | | |
| | 3-like 4 | | | | | | |
| NM_016823.2 | v-crk sarcoma | CRK | 2% | 10% | 1.91 | 1.47 | 9% |
| | virus CT10 | | | | | | |
| | oncogene | | | | | | |
| | homolog | | | | | | |
| | (avian) | | | | | | |
| NM_177559.2 | casein kinase 2, | CSNK2A1 | 6% | 12% | 2.03 | 1.93 | 7% |
| | alpha 1 | | | | | | |
| | polypeptide | | | | | | |
| NM_004078.1 | cysteine and | CSRP1 | 0% | 6% | 1.00 | 2.54 | 8% |
| | glycine-rich | | | | | | |
| | protein 1 | | | | | | |
| NM_018959.1 | DAZ associated | DAZAP1 | 2% | 8% | 2.10 | 2.13 | 8% |
| | protein 1 | | | | | | |
| BC001286.1 | dCMP | DCTD | 4% | 12% | 1.58 | 1.69 | 10% |
| | deaminase | | | | | | |
| NM_001930.2 | deoxyhypusine | DHPS | 2% | 10% | 1.42 | 3.62 | 13% |
| | synthase | | | | | | |
| NM_138930.2 | diablo homolog | DIABLO | 2% | 10% | 1.16 | 1.75 | 10% |
| | (Drosophila) | | | | | | |
| BC069363.1 | distal-less | DLX6 | 0% | 6% | 1.00 | 2.76 | 8% |
| | homeobox 6 | | | | | | |
| BC033159.1 | DnaJ (Hsp40) | DNAJC8 | 0% | 4% | 1.00 | 2.66 | 5% |
| | homolog, | | | | | | |
| | subfamily C, | | | | | | |
| | member 8 | | | | | | |
| NM_006442.2 | DR1-associated | DRAP1 | 0% | 14% | 1.00 | 2.07 | 18% |
| | protein 1 | | | | | | |
| | (negative | | | | | | |
| | cofactor 2 | | | | | | |
| | alpha) | | | | | | |
| NM_003746.1 | dynein, light | DYNLL1 | 0% | 4% | 1.00 | 3.19 | 6% |
| | chain, LC8- | | | | | | |
| | type 1 | | | | | | |
| BC013648.1 | EF hand | EFHD2 | 0% | 6% | 1.00 | 1.71 | 7% |
| | domain family, | | | | | | |
| | member D2 | | | | | | |
| NM_003754.1 | eukaryotic | EIF3S5 | 4% | 12% | 3.47 | 1.65 | 8% |
| | translation | | | | | | |
| | initiation factor | | | | | | |
| | 3, subunit 5 | | | | | | |
| | epsilon, 47 kDa | | | | | | |
| NM_005232.1 | EPH receptor | EPHA1 | 2% | 12% | 1.01 | 3.01 | 15% |
| | A1 | | | | | | |
| NM_004441.2 | EPH receptor | EPHB1 | 0% | 8% | 1.00 | 2.55 | 11% |
| | B1 | | | | | | |
| NM_018948.2 | ERBB receptor | ERRFI1 | 0% | 10% | 1.00 | 3.43 | 15% |
| | feedback | | | | | | |
| | inhibitor 1 | | | | | | |
| BC050543.1 | family with | FAM131B | 4% | 12% | 2.78 | 1.79 | 9% |
| | sequence | | | | | | |
| | similarity 131, | | | | | | |
| | member B | | | | | | |
| NM_152789.1 | family with | FAM133B | 2% | 6% | 1.04 | 4.53 | 8% |
| | sequence | | | | | | |
| | similarity 133, | | | | | | |
| | member B | | | | | | |
| NM_152421.2 | family with | FAM69B | 0% | 6% | 1.00 | 2.09 | 8% |
| | sequence | | | | | | |
| | similarity 69, | | | | | | |
| | member B | | | | | | |
| NM_018145.1 | family with | FAM82C | 2% | 10% | 2.17 | 1.79 | 9% |
| | sequence | | | | | | |
| | similarity 82, | | | | | | |
| | member C | | | | | | |
| NM_005246.1 | fer (fps/fes | FER | 0% | 12% | 1.00 | 2.77 | 17% |
| | related) | | | | | | |
| | tyrosine kinase | | | | | | |
| | (phosphoprotein | | | | | | |
| | NCP94) | | | | | | |
| NM_015850.2 | fibroblast | FGFR1 | 4% | 12% | 3.20 | 7.16 | 17% |
| | growth factor | | | | | | |
| | receptor 1 | | | | | | |
| | (fms-related | | | | | | |
| | tyrosine kinase | | | | | | |
| | 2, Pfeiffer | | | | | | |
| | syndrome) | | | | | | |
| BC000084.1 | hypothetical | FLJ10357 | 2% | 8% | 3.02 | 1.49 | 6% |
| | protein | | | | | | |
| | FLJ10357 | | | | | | |
| BC021906.1 | formin-like 1 | FMNL1 | 2% | 6% | 1.05 | 6.05 | 9% |
| NM_144769.1 | forkhead box I1 | FOXI1 | 2% | 8% | 1.19 | 3.87 | 10% |
| BC009642.1 | FXYD domain | FXYD5 | 0% | 10% | 1.00 | 1.76 | 12% |
| | containing ion | | | | | | |
| | transport | | | | | | |
| | regulator 5 | | | | | | |
| BC008668.1 | cyclin G | GAK | 6% | 12% | 1.34 | 3.25 | 11% |
| | associated | | | | | | |
| | kinase | | | | | | |
| NM_177553.1 | growth arrest- | GAS2 | 0% | 4% | 1.00 | 3.70 | 6% |
| | specific 2 | | | | | | |
| NM_201432.1 | growth arrest- | GAS7 | 2% | 12% | 1.01 | 2.15 | 13% |
| | specific 7 | | | | | | |
| NM_178044.1 | GIY-YIG | GIYD2 | 0% | 4% | 1.00 | 5.26 | 7% |
| | domain | | | | | | |
| | containing 2 | | | | | | |
| BC015848.1 | glyoxalase | GLOD4 | 0% | 8% | 1.00 | 1.42 | 9% |
| | domain | | | | | | |
| | containing 4 | | | | | | |
| NM_033003.1 | general | GTF2I | 0% | 6% | 1.00 | 3.14 | 9% |
| | transcription | | | | | | |
| | factor II, i | | | | | | |
| NM_138612.1 | hyaluronan | HAS3 | 2% | 6% | 1.87 | 2.20 | 5% |
| | synthase 3 | | | | | | |
| BC021278.1 | hexokinase | HKDC1 | 0% | 8% | 1.00 | 2.67 | 11% |
| | domain | | | | | | |
| | containing 1 | | | | | | |
| BC008959.1 | histocompatibility | HM13 | 0% | 8% | 1.00 | 6.89 | 15% |
| | (minor) 13 | | | | | | |
| NM_002128.2 | high-mobility | HMGB1 | 4% | 10% | 2.29 | 2.15 | 8% |
| | group box 1 | | | | | | |
| BC015358.1 | HIV-1 Tat | HTATIP2 | 2% | 6% | 0.78 | 3.46 | 7% |
| | interactive | | | | | | |
| | protein 2, | | | | | | |
| | 30 kDa | | | | | | |
| NM_033261.2 | isopentenyl- | IDI2 | 2% | 6% | 1.11 | 2.01 | 5% |
| | diphosphate | | | | | | |
| | delta isomerase 2 | | | | | | |
| NM_021803.1 | interleukin 21 | IL21 | 0% | 6% | 1.00 | 2.52 | 8% |
| NM_173192.1 | Kv channel | KCNIP2 | 4% | 10% | 1.65 | 1.61 | 7% |
| | interacting | | | | | | |
| | protein 2 | | | | | | |
| BC025726.1 | potassium | KCNK17 | 0% | 8% | 1.00 | 1.55 | 9% |
| | channel, | | | | | | |
| | subfamily K, | | | | | | |
| | member 17 | | | | | | |
| NM_024076.1 | potassium | KCTD15 | 0% | 10% | 1.00 | 2.60 | 13% |
| | channel | | | | | | |
| | tetramerisation | | | | | | |
| | domain | | | | | | |
| | containing 15 | | | | | | |
| BC052802.1 | KIAA0467 | KIAA0467 | 2% | 8% | 1.12 | 2.03 | 8% |
| BC017355.1 | Karyopherin | KPNA3 | 6% | 16% | 1.23 | 2.16 | 14% |
| | alpha 3 | | | | | | |
| | (importin alpha 4) | | | | | | |
| BC021241.2 | keratin 81 | KRT81 | 2% | 8% | 1.05 | 1.70 | 7% |
| BC001370.2 | LIM and | LIMS2 | 2% | 6% | 1.30 | 4.15 | 7% |
| | senescent cell | | | | | | |
| | antigen-like | | | | | | |
| | domains 2 | | | | | | |
| BC066353.1 | LIM homeobox | LMX1A | 2% | 8% | 4.03 | 2.48 | 8% |
| | transcription | | | | | | |
| | factor 1, alpha | | | | | | |
| BC062736.1 | basic | LOC503543 | 0% | 6% | 1.00 | 1.91 | 7% |
| | transcription | | | | | | |
| | factor 3, | | | | | | |
| | pseudogene 9 | | | | | | |
| NM_138353.1 | hypothetical | LOC90379 | 2% | 14% | 1.09 | 1.77 | 15% |
| | protein | | | | | | |
| | BC002926 | | | | | | |
| BC003408.2 | melanoma | MAGEA12 | 2% | 8% | 1.39 | 2.79 | 9% |
| | antigen family | | | | | | |
| | A, 12 | | | | | | |
| NM_002362.2 | melanoma | MAGEA4 | 0% | 4% | 1.00 | 29.91 | 12% |
| | antigen family | | | | | | |
| | A, 4 | | | | | | |
| NM_002364.1 | melanoma | MAGEB2 | 4% | 10% | 7.67 | 2.61 | 6% |
| | antigen family | | | | | | |
| | B, 2 | | | | | | |
| NM_032332.2 | mitogen- | MORG1 | 2% | 8% | 1.02 | 1.69 | 7% |
| | activated | | | | | | |
| | protein kinase | | | | | | |
| | organizer 1 | | | | | | |
| NM_002753.2 | mitogen- | MAPK10 | 0% | 6% | 1.00 | 2.84 | 8% |
| | activated | | | | | | |
| | protein kinase 10 | | | | | | |
| NM_138993.1 | mitogen- | MAPK11 | 2% | 10% | 1.25 | 4.42 | 14% |
| | activated | | | | | | |
| | protein kinase 11 | | | | | | |
| NM_004635.2 | mitogen- | MAPKAPK3 | 0% | 10% | 1.00 | 1.81 | 12% |
| | activated | | | | | | |
| | protein kinase- | | | | | | |
| | activated | | | | | | |
| | protein kinase 3 | | | | | | |
| NM_003668.2 | mitogen- | MAPKAPK5 | 2% | 10% | 14.49 | 3.70 | 11% |
| | activated | | | | | | |
| | protein kinase- | | | | | | |
| | activated | | | | | | |
| | protein kinase 5 | | | | | | |
| NM_032601.2 | methylmalonyl | MCEE | 4% | 10% | 1.11 | 2.56 | 10% |
| | CoA epimerase | | | | | | |
| BC009398.1 | minichromosome | MCM7 | 0% | 6% | 1.00 | 1.52 | 7% |
| | maintenance | | | | | | |
| | deficient (S. cerevisiae) 7 | | | | | | |
| BC006005.1 | uncharacterized | MDS032 | 2% | 8% | 1.34 | 1.81 | 7% |
| | hematopoietic | | | | | | |
| | stem/progenitor | | | | | | |
| | cells protein | | | | | | |
| | MDS032 | | | | | | |
| NM_006343.1 | c-mer proto- | MERTK | 2% | 10% | 1.11 | 3.06 | 12% |
| | oncogene | | | | | | |
| | tyrosine kinase | | | | | | |
| BC028039.1 | hypothetical | MGC39900 | 4% | 12% | 2.05 | 1.85 | 10% |
| | protein | | | | | | |
| | MGC39900 | | | | | | |
| NM_001004306.1 | similar to | MGC87631 | 2% | 8% | 1.01 | 2.13 | 8% |
| | hypothetical | | | | | | |
| | protein | | | | | | |
| | FLJ36492 | | | | | | |
| NM_015246.1 | mahogunin, | MGRN1 | 0% | 6% | 1.00 | 2.33 | 8% |
| | ring finger 1 | | | | | | |
| NM_017572.1 | MAP kinase | MKNK2 | 2% | 6% | 1.25 | 2.70 | 6% |
| | interacting | | | | | | |
| | serine/threonine | | | | | | |
| | kinase 2 | | | | | | |
| NM_198204.1 | MAX-like | MLX | 2% | 12% | 1.75 | 2.15 | 13% |
| | protein X | | | | | | |
| NM_020236.2 | mitochondrial | MRPL1 | 2% | 6% | 2.11 | 2.46 | 6% |
| | ribosomal | | | | | | |
| | protein L1 | | | | | | |
| BC052601.1 | mitochondrial | MRPL10 | 0% | 6% | 1.00 | 2.42 | 8% |
| | ribosomal | | | | | | |
| | protein L10 | | | | | | |
| NM_006428.3 | mitochondrial | MRPL28 | 2% | 12% | 3.01 | 1.94 | 12% |
| | ribosomal | | | | | | |
| | protein L28 | | | | | | |
| BC030521.2 | mitochondrial | MRPS27 | 0% | 6% | 1.00 | 3.28 | 9% |
| | ribosomal | | | | | | |
| | protein S27 | | | | | | |
| NM_007067.3 | MYST histone | MYST2 | 0% | 14% | 1.00 | 3.08 | 20% |
| | acetyltransferase 2 | | | | | | |
| NM_005000.2 | NADH | NDUFA5 | 2% | 10% | 2.98 | 3.83 | 13% |
| | dehydrogenase | | | | | | |
| | (ubiquinone) 1 | | | | | | |
| | alpha | | | | | | |
| | subcomplex, 5, | | | | | | |
| | 13 kDa | | | | | | |
| NM_002505.3 | nuclear | NFYA | 2% | 10% | 1.21 | 2.80 | 12% |
| | transcription | | | | | | |
| | factor Y, alpha | | | | | | |
| NM_006993.1 | nucleophosmin/ | NPM3 | 4% | 16% | 1.44 | 2.65 | 17% |
| | nucleoplasmin 3 | | | | | | |
| NM_052935.2 | 5′-nucleotidase, | NT5C3L | 0% | 4% | 1.00 | 3.30 | 6% |
| | cytosolic III- | | | | | | |
| | like | | | | | | |
| BC010176.1 | sarcoma | NY-SAR-48 | 0% | 8% | 1.00 | 2.70 | 11% |
| | antigen NY- | | | | | | |
| | SAR-48 | | | | | | |
| BC009779.1 | outer dense | ODF2L | 0% | 4% | 1.00 | 2.30 | 5% |
| | fiber of sperm | | | | | | |
| | tails 2-like | | | | | | |
| NM_012373.1 | olfactory | OR3A3 | 4% | 10% | 2.33 | 1.73 | 7% |
| | receptor, family | | | | | | |
| | 3, subfamily A, | | | | | | |
| | member 3 | | | | | | |
| NM_014562.2 | orthodenticle | OTX1 | 0% | 4% | 1.00 | 2.75 | 5% |
| | homeobox 1 | | | | | | |
| NM_001014831.1 | p21(CDKN1A)- | PAK4 | 2% | 10% | 1.47 | 1.57 | 9% |
| | activated | | | | | | |
| | kinase 4 | | | | | | |
| NM_148978.1 | pantothenate | PANK1 | 2% | 8% | 1.32 | 4.94 | 11% |
| | kinase 1 | | | | | | |
| BC039742.1 | poly(rC) | PCBP1 | 4% | 8% | 1.07 | 1.64 | 5% |
| | binding protein 1 | | | | | | |
| NM_002591.2 | phosphoenolpyruvate | PCK1 | 4% | 10% | 1.32 | 3.63 | 11% |
| | carboxykinase | | | | | | |
| | 1 (soluble) | | | | | | |
| NM_030948.1 | phosphatase | PHACTR1 | 2% | 8% | 1.82 | 2.95 | 9% |
| | and actin | | | | | | |
| | regulator 1 | | | | | | |
| BC013031.1 | pleckstrin | PHLDB1 | 2% | 10% | 1.20 | 1.79 | 10% |
| | homology-like | | | | | | |
| | domain, family | | | | | | |
| | B, member 1 | | | | | | |
| BC030815.1 | phosphoinositide- | PIK3R1 | 0% | 6% | 1.00 | 1.67 | 7% |
| | 3-kinase, | | | | | | |
| | regulatory | | | | | | |
| | subunit 1 (p85 | | | | | | |
| | alpha) | | | | | | |
| NM_001722.2 | polymerase | POLR3D | 0% | 6% | 1.00 | 2.12 | 8% |
| | (RNA) III | | | | | | |
| | (DNA directed) | | | | | | |
| | polypeptide D, | | | | | | |
| | 44 kDa | | | | | | |
| NM_004227.3 | pleckstrin | PSCD3 | 0% | 10% | 1.00 | 1.48 | 11% |
| | homology, | | | | | | |
| | Sec7 and | | | | | | |
| | coiled/coil | | | | | | |
| | domains 3 | | | | | | |
| NM_170750.1 | proteasome | PSMD10 | 2% | 10% | 1.54 | 1.35 | 9% |
| | (prosome, | | | | | | |
| | macropain) 26S | | | | | | |
| | subunit, non- | | | | | | |
| | ATPase, 10 | | | | | | |
| BC001772.1 | glutaminyl- | QARS | 2% | 8% | 1.53 | 2.02 | 8% |
| | tRNA | | | | | | |
| | synthetase | | | | | | |
| NM_030981.1 | RAB1B, | RAB1B | 4% | 8% | 1.22 | 2.13 | 6% |
| | member RAS | | | | | | |
| | oncogene | | | | | | |
| | family | | | | | | |
| BC002510.2 | RAB6B, | RAB6B | 0% | 4% | 1.00 | 4.11 | 6% |
| | member RAS | | | | | | |
| | oncogene | | | | | | |
| | family | | | | | | |
| NM_005053.2 | RAD23 | RAD23A | 4% | 14% | 1.42 | 2.66 | 15% |
| | homolog A (S. cerevisiae) | | | | | | |
| NM_005105.2 | RNA binding | RBM8A | 6% | 14% | 1.79 | 1.55 | 9% |
| | motif protein | | | | | | |
| | 8A | | | | | | |
| BC013115.1 | RNA binding | RBM9 | 0% | 6% | 1.00 | 2.08 | 8% |
| | motif protein 9 | | | | | | |
| NM_016606.2 | receptor | REEP2 | 0% | 6% | 1.00 | 2.48 | 8% |
| | accessory | | | | | | |
| | protein 2 | | | | | | |
| NM_006480.4 | regulator of G- | RGS14 | 0% | 6% | 1.00 | 2.39 | 8% |
| | protein | | | | | | |
| | signalling 14 | | | | | | |
| BC043348.2 | retinitis | RP2 | 4% | 10% | 1.42 | 2.16 | 8% |
| | pigmentosa 2 | | | | | | |
| | (X-linked | | | | | | |
| | recessive) | | | | | | |
| NM_002946.2 | replication | RPA2 | 2% | 10% | 1.02 | 3.68 | 13% |
| | protein A2, | | | | | | |
| | 32 kDa | | | | | | |
| NM_015414.2 | ribosomal | RPL36 | 2% | 8% | 4.37 | 1.77 | 6% |
| | protein L36 | | | | | | |
| BC001697.1 | ribosomal | RPS15A | 2% | 8% | 1.02 | 2.25 | 8% |
| | protein S15a | | | | | | |
| BC014959.1 | RING1 and | RYBP | 0% | 6% | 1.00 | 2.48 | 8% |
| | YY1 binding | | | | | | |
| | protein | | | | | | |
| NM_005620.1 | S100 calcium | S100A11 | 0% | 10% | 1.00 | 1.67 | 12% |
| | binding protein | | | | | | |
| | A11 | | | | | | |
| NM_005621.1 | S100 calcium | S100A12 | 0% | 8% | 1.00 | 2.05 | 10% |
| | binding protein | | | | | | |
| | A12 | | | | | | |
| BC024245.2 | sal-like 2 | SALL2 | 0% | 8% | 1.00 | 1.85 | 10% |
| | (Drosophila) | | | | | | |
| BC041638.1 | squamous cell | SART3 | 0% | 8% | 1.00 | 4.48 | 13% |
| | carcinoma | | | | | | |
| | antigen | | | | | | |
| | recognised by | | | | | | |
| | T cells 3 | | | | | | |
| NM_005698.2 | secretory | SCAMP3 | 2% | 8% | 1.31 | 1.77 | 7% |
| | carrier | | | | | | |
| | membrane | | | | | | |
| | protein 3 | | | | | | |
| NM_020423.1 | SCY1-like 3 (S. cerevisiae) | SCYL3 | 0% | 8% | 1.00 | 3.11 | 11% |
| BC000463.1 | splicing factor | SF3B3 | 2% | 8% | 1.21 | 2.49 | 9% |
| | 3b, subunit 3, | | | | | | |
| | 130 kDa | | | | | | |
| NM_144595.1 | hypothetical | FLJ30046 | 0% | 4% | 1.00 | 3.71 | 6% |
| | protein | | | | | | |
| | FLJ30046 | | | | | | |
| NM_005901.2 | SMAD family | SMAD2 | 2% | 12% | 2.94 | 2.15 | 12% |
| | member 2 | | | | | | |
| NM_005902.1 | SMAD family | SMAD3 | 0% | 6% | 1.00 | 3.38 | 9% |
| | member 3 | | | | | | |
| NM_004782.2 | synaptosomal- | SNAP29 | 0% | 4% | 1.00 | 3.44 | 6% |
| | associated | | | | | | |
| | protein, 29 kDa | | | | | | |
| NM_015464.1 | sclerostin | SOSTDC1 | 0% | 6% | 1.00 | 4.84 | 10% |
| | domain | | | | | | |
| | containing 1 | | | | | | |
| NM_021203.2 | signal | SRPRB | 0% | 6% | 1.00 | 1.47 | 7% |
| | recognition | | | | | | |
| | particle | | | | | | |
| | receptor, B | | | | | | |
| | subunit | | | | | | |
| BC007919.2 | START domain | STARD10 | 2% | 10% | 1.32 | 2.24 | 11% |
| | containing 10 | | | | | | |
| NM_006374.2 | serine/threonine | STK25 | 0% | 6% | 1.00 | 1.40 | 7% |
| | kinase 25 | | | | | | |
| | (STE20 | | | | | | |
| | homolog, | | | | | | |
| | yeast) | | | | | | |
| NM_178509.3 | syntaxin | STXBP4 | 2% | 6% | 2.21 | 9.07 | 10% |
| | binding protein 4 | | | | | | |
| BC010537.1 | SUB1 homolog | SUB1 | 0% | 10% | 1.00 | 1.50 | 11% |
| | (S. cerevisiae) | | | | | | |
| NM_003352.4 | SMT3 | SUMO1 | 2% | 10% | 1.31 | 4.18 | 14% |
| | suppressor of | | | | | | |
| | mif two 3 | | | | | | |
| | homolog 1 (S. cerevisiae) | | | | | | |
| BC022340.1 | SMT3 | SUMO2 | 2% | 6% | 1.10 | 2.83 | 6% |
| | suppressor of | | | | | | |
| | mif two 3 | | | | | | |
| | homolog 2 (S. cerevisiae) | | | | | | |
| NM_153694.3 | synaptonemal | SYCP3 | 0% | 6% | 1.00 | 1.99 | 7% |
| | complex | | | | | | |
| | protein 3 | | | | | | |
| NM_013342.1 | TCF3 (E2A) | TFPT | 0% | 6% | 1.00 | 1.49 | 7% |
| | fusion partner | | | | | | |
| | (in childhood | | | | | | |
| | Leukemia) | | | | | | |
| NM_021809.2 | TGFB-induced | TGIF2 | 4% | 10% | 1.62 | 12.63 | 18% |
| | factor | | | | | | |
| | homeobox 2 | | | | | | |
| BC029920.1 | TGFB-induced | TGIF2LX | 4% | 10% | 1.77 | 27.12 | 25% |
| | factor | | | | | | |
| | homeobox 2- | | | | | | |
| | like, X-linked | | | | | | |
| BC001852.2 | tRNA-histidine | THG1L | 2% | 10% | 1.99 | 1.79 | 10% |
| | guanylyltransferase | | | | | | |
| | 1-like (S. cerevisiae) | | | | | | |
| NM_138461.1 | transmembrane | TM4SF19 | 2% | 6% | 1.06 | 2.17 | 6% |
| | 4 L six family | | | | | | |
| | member 19 | | | | | | |
| NM_145041.1 | transmembrane | TMEM106A | 2% | 12% | 1.36 | 2.03 | 13% |
| | protein 106A | | | | | | |
| BC002660.1 | tropomodulin 1 | TMOD1 | 0% | 6% | 1.00 | 3.75 | 9% |
| BC053675.1 | thymopoietin | TMPO | 4% | 10% | 2.25 | 5.23 | 12% |
| NM_021103.2 | thymosin, beta 10 | TMSB10 | 2% | 10% | 1.08 | 2.67 | 12% |
| NM_032174.2 | translocase of | TOMM40L | 4% | 8% | 1.15 | 3.57 | 8% |
| | outer | | | | | | |
| | mitochondrial | | | | | | |
| | membrane 40 | | | | | | |
| | homolog-like | | | | | | |
| | (yeast) | | | | | | |
| BC003596.1 | tumor protein | TP53 | 0% | 10% | 1.00 | 4.82 | 17% |
| | p53 (Li- | | | | | | |
| | Fraumeni | | | | | | |
| | syndrome) | | | | | | |
| BC070290.1 | triadin | TRDN | 2% | 6% | 1.10 | 3.66 | 7% |
| NM_007117.1 | thyrotropin- | TRH | 0% | 10% | 1.00 | 1.89 | 12% |
| | releasing | | | | | | |
| | hormone | | | | | | |
| NM_004240.2 | thyroid | TRIP10 | 2% | 10% | 2.17 | 1.91 | 10% |
| | hormone | | | | | | |
| | receptor | | | | | | |
| | interactor 10 | | | | | | |
| NM_139169.2 | TruB | TRUB1 | 2% | 12% | 1.43 | 2.41 | 14% |
| | pseudouridine | | | | | | |
| | (psi) synthase | | | | | | |
| | homolog 1 (E. coli) | | | | | | |
| NM_030935.1 | TSC22 domain | TSC22D4 | 2% | 12% | 1.08 | 8.69 | 22% |
| | family, member 4 | | | | | | |
| NM_130465.3 | tetraspanin 17 | TSPAN17 | 6% | 14% | 6.36 | 1.98 | 7% |
| NM_015959.1 | thioredoxin | TXNDC14 | 0% | 4% | 1.00 | 4.70 | 7% |
| | domain | | | | | | |
| | containing 14 | | | | | | |
| NM_181892.1 | ubiquitin- | UBE2D3 | 2% | 8% | 1.13 | 1.89 | 8% |
| | conjugating | | | | | | |
| | enzyme E2D 3 | | | | | | |
| | (UBC4/5 | | | | | | |
| | homolog, | | | | | | |
| | yeast) | | | | | | |
| NM_003348.3 | ubiquitin- | UBE2N | 2% | 8% | 1.16 | 2.52 | 9% |
| | conjugating | | | | | | |
| | enzyme E2N | | | | | | |
| | (UBC13 | | | | | | |
| | homolog, | | | | | | |
| | yeast) | | | | | | |
| BC043346.2 | ubiquitin-like | UBL4A | 2% | 8% | 1.07 | 13.11 | 17% |
| | 4A | | | | | | |
| NM_152277.1 | dendritic cell- | DC-UbP | 6% | 24% | 1.82 | 3.93 | 30% |
| | derived | | | | | | |
| | ubiquitin-like | | | | | | |
| | protein | | | | | | |
| NM_173517.3 | vitamin K | VKORC1L1 | 0% | 4% | 1.00 | 24.50 | 11% |
| | epoxide | | | | | | |
| | reductase | | | | | | |
| | complex, | | | | | | |
| | subunit 1-like 1 | | | | | | |
| BC001001.2 | vacuolar | VPS8 | 6% | 12% | 1.78 | 1.87 | 8% |
| | protein sorting | | | | | | |
| | 8 homolog (S. cerevisiae) | | | | | | |
| BC023556.2 | vaccinia related | VRK3 | 0% | 6% | 1.00 | 2.50 | 8% |
| | kinase 3 | | | | | | |
| NM_015671.2 | WD repeat | WDR62 | 0% | 4% | 1.00 | 4.71 | 7% |
| | domain 62 | | | | | | |
| NM_003404.2 | tyrosine 3- | YWHAB | 0% | 8% | 1.00 | 3.74 | 12% |
| | monooxygenase/ | | | | | | |
| | tryptophan 5- | | | | | | |
| | monooxygenase | | | | | | |
| | activation | | | | | | |
| | protein, beta | | | | | | |
| | polypeptide | | | | | | |
| NM_017612.1 | zinc finger, | ZCCHC8 | 2% | 8% | 1.48 | 2.76 | 9% |
| | CCHC domain | | | | | | |
| | containing 8 | | | | | | |
| NM_015363.3 | zinc finger, | ZIM2 | 2% | 10% | 1.36 | 3.80 | 13% |
| | imprinted 2 | | | | | | |
| BC000876.1 | zinc finger | ZNF174 | 2% | 10% | 1.21 | 1.54 | 9% |
| | protein 174 | | | | | | |
| BC002859.1 | zinc finger | ZNF434 | 0% | 14% | 1.00 | 1.71 | 16% |
| | protein 434 | | | | | | |

### EXAMPLES

### Example 1

### Materials and Methods

### Ovarian Patient Samples:

Serum samples were obtained with approved consent from 52 epithelial ovarian cancer (EOC) patients at the time of surgery at Roswell Park Cancer Institute under an Institutional Review Board approved protocol and from 53 healthy age-matched donors from the New York blood bank and from Roswell Park Cancer Institute. Patient characteristics are described in Table 1.

Of the ovarian cancer patients examined, 29 (57%) were dead before the end of the observation period. The median duration of follow-up for the entire group was 47.7 months (range, 1.0-167 months). The median age of the study population was 62 years (range, 26-88 years). The majority of patients presented with grade-3 tumors (82%), at stage IIIC (86%), and with serous histology (82%). The median survival for all patients was 48 months (95% confidence interval (C.I.)=27−infinity months). The 5-year survival for the entire study population was 45%. The ovarian cancer patients were selected to include 26 long-term survivors (median survival=74 months), 7 intermediate-term survivors (median survival=27 months), and 18 short-term survivors (median survival=8 months). The control sample (n=53) had a median age of 62 years (range, 38-92 years) and was 49% female. The healthy control serum samples were from the Data Bank and BioRepository of Roswell Park Cancer Institute (37, or 70%) and from the New York Blood Bank (16, or 30%). Healthy age-matched donors characteristics are described in Table 2.

### ELISA:

Patient or donor serum samples were analyzed by ELISA for seroreactivity to bacterially-produced recombinant proteins NY-ESO-1/CTAG1B, LAGE-1/CTAG2, MAGEA1, MAGEA3, MAGEA4, MAGEA10, CT7/MAGEC1, CT10/MAGEC2, CT45/RP13-36C9.1, CT46/HORMAD1, CT47/RP6-166C19.1, Ki-67/MKI67, KRAS, SCP1/SYCP1, SOX2, SPANXA1, SSX1, SSX2, SSX4, p53/TP53, XAGE1B, and DHFR (Atanackovic et al, PNAS 2008). Serum was diluted serially from 1/100 to 1/100,000 and added to low-volume 96-well plates (Corning, N.Y.) coated overnight at 4° C. with 1 μg/ml antigen in 25 μl volume and blocked for 2 h at RT with PBS containing 5% non-fat milk. After overnight incubation, plates were extensively washed with PBS containing 0.2% Tween 20 and rinsed with PBS (BioTek ELx405 automated washer, Winooski, Vt.). Plasma IgG (total or subclasses) bound to antigens was detected with specific monoclonal antibodies conjugated with alkaline-phosphatase (Southern Biotech, Birmingham, Ala.). Following addition of ATTOPHOS substrate (Fisher Scientific, Waltham, Mass.), absorbance was measured using a fluorescence reader Cytofluor Series 4000 (PerSeptive Biosystems, Framingham, Mass.). A reciprocal titer was calculated for each plasma sample as the maximal dilution still significantly reacting to a specific antigen. Specificity was determined by comparing seroreactivity among various antigens tested. In each assay, sera of patients with known presence or absence of specific reactivity were used as controls. A positive result was defined as extrapolated reciprocal titers >100.

### Protein Microarray:

ProtoArrays® microarrays (v4.0; Invitrogen, Carlsbad) were purchased and used according to the manufacturer's instructions.

### Array Profiling Assay:

Arrays were incubated in recommended blocking buffer (50 mM HEPES, pH 7.5, 200 mM NaCl, 0.08% Triton® X-100, 25% Glycerol, 20 mM Reduced glutathione, 1× Roti®-Block, 1 mM DTT) for 1 h at 4° C. in Quadriperm dishes (Greiner BioOne, Stonehouse, UK) placed on a horizontal shaker (50 rpm). Arrays were then incubated for 90 min at 4° C. with individual sera diluted at 1:500 in 4 ml buffer (1×PBS, 0.1% Tween 20, 1× Roti®-Block). After washes, binding of IgG was detected by incubation with Alexa Fluor® 647 anti-human IgG (Invitrogen) diluted 1:2000 in assay buffer for 90 min at 4° C. Arrays were washed again and dried by centrifugation. Arrays were scanned at 10 μm resolution using a microarray scanner (Axon 4200AL with GenePix Pro Software, Molecular Devices, Sunnyvale, Calif. 94089) and fluorescence detected according to the manufacturer's instructions. Images were saved as 16-bit tif files and analysis was performed using GenePix. The mean and median net intensity in relative fluorescence units (rfu) were reported for each spot.

### Calculations:

Data from arrays were adjusted and normalized in the following sequence. First, minimal median or mean rfu values of duplicate spots were averaged for each antigen within each array. Next, for each array, values for each antigen were expressed as a percentage of interquartile differences in relation to all other antigens on the array using the following formula:

$$\frac{( Observed value ) - ( {25}^{th} percentile )}{( {75}^{th} percentile ) - ( {25}^{th} percentile )} .$$

Finally, all percentage values were normalized using a standard quantile normalization matrix (Bolstad et al., 2003, Bioinformatics 19, 185-193), in which all percentage values of each array were ranked and replaced by the average of percentages for antigens with the same rank on all arrays. These successive transformations resulted in a data distribution with identical median and quartile values, to allow interarray comparisons.

Once data on arrays was normalized, the interquartile difference was calculated for each antigen across all arrays. These interquartile values were used to establish a cutoff and determine antigens showing significant seroreactivity: to be defined as positive for a given antigen, a serum had to react to the antigen with values greater than 2.5× the interquartile difference above the 75^{th} percentile. These stringent criteria were used to minimize false positive data while providing increased specificity and sensitivity. If the reaction of a serum to an antigen is greater than the cutoff, the ratio between signal and cutoff (S/C ratio) is calculated. The more reactive the serum, the higher the ratio of signal to cutoff is for each antigen.

Finally, a score was assigned taking into account the strength and frequency of signal in healthy donor sera compared to cancer patient sera. To this end, the frequency of sera responding to each antigen was determined by counting how many samples displayed signals above cutoff, independently for healthy donors and lung cancer patients. Then, an average of S/C ratio was calculated per antigen for each cohort, to provide an overall estimate of the strength of reactivity observed. In order to avoid overemphasizing individual rare sera with very high responses, the contribution of the signal strength (S/C ratio) score was reduced by taking its cubic root. Finally, for each antigen, the frequency of responders in each cohort was multiplied by the cubic root of S/C ratio averages. Therefore, emphasis was placed on the frequency in each cohort, while still considering strength of signal. For example, if 10% of healthy donors have a response to an antigen, but if most of these responses are only barely higher than the cutoff and therefore weak, they will have a lower score compared to 10% of lung cancer patient sera with high S/C ratios for the same antigen. The final ranking of reactive antigens was determined by the difference between the scores in each cohort (patient—healthy). A high score (>5%) indicates that most seroreactivity is attributable to cancer patients. For ELISA versus seromics comparisons, the Pearson correlation test was used. For clinical correlations of overall survival with the presence of specific antibodies, results were analyzed by the Kaplan-Meier method and assessed for significance using the log-rank method.

### Array Analyses:

We describe the use of protein microarrays containing >8200 full-length protein antigens for the detection of serum antibody responses in cancer patients. We validated the use of these microarrays with serum samples from ovarian cancer patients and healthy donors previously tested by ELISA for reactivity against selected recombinant tumor antigens present on the arrays. Importantly, we describe a set of simple data analysis tools, from normalization to scoring methods, that were specifically designed to address the question of specificity of antibody responses in proteomic approaches and yet have a universal applicability to other types of protein arrays.

Contrary to widely used normalization and biostatistical methods for analyzing microarray data in the genomics field, our methods take into account the unique challenges presented by the use of serum as a probe, more variable in nature than DNA or RNA. Accordingly, when a typical genomic data analysis looks for small but consistent changes across two data sets, our analysis method highlights relatively rare events with high significance (corresponding to sera reacting with high titers for a specific antigen) and ranks them with biological relevance for the cancer cohort.

This work defines antigenic targets for cancer vaccine development, serum antibody signatures with clinical value, characterization of predictive serum markers for experimental therapeutics.

### Example 2

### Measuring Autoantibodies to Known Tumor Antigens by ELISA

To establish a profile of autoantibody reactivity to tumor antigens, we tested a panel of 22 recombinant proteins, including cancer/testis (CT) antigens (NY-ESO-1, LAGE-1, MAGE-A, MAGE-C, SSX, XAGE, CT45, CT46, CT47, SPANX), mutational antigens (TP53, K-ras), and embryonic/stem cell antigens (SOX2), for recognition by ELISA. As expected from previous studies, NY-ESO-1 and LAGE-1 were recognized with the highest frequency by ovarian cancer sera (17% and 19%, respectively). Serum reactivity to MAGE antigens, SSX2, CT7, or CT10 was also found in ovarian cancer samples. Overall, antibody responses to one or more antigens from the panel were found in 52% of ovarian cancer samples. Except for NY-ESO-1 and LAGE-1, all other antigens tested reacted with less than 10% of sera within each cohort, indicating that detectable spontaneous immunogenicity to these antigens represents an uncommon event. Ovarian cancer sera were more likely to react to multiple antigens simultaneously, and did so with higher average titers compared to sera from healthy donors.

### Example 3

### Validating the Use of Protein Microarrays

Having previously established that protein microarrays (including ProtoArrays) were a suitable method for detecting antibody responses from the serum of non-small-cell lung cancer patients, we reexamined the concordance between ELISA and seromics for the current investigation. Because only two of the proteins tested in ELISA, MAGE-A4 and TP53, were present on ProtoArrays, we sought to extend the correlation between assays by spotting most of the panel of recombinant proteins used for ELISAs in a customized fashion on ProtoArrays, along with additional control proteins.

All sera were tested at a dilution of 1:500 as described in Materials and Methods, and antigen-specific IgG responses to each of the proteins present on the array were measured by fluorescence. Reproducibility of results was confirmed using duplicate microarrays for selected sera. After extensive visual quality control of spot alignment and duplicates, a series of normalization steps was applied to allow interslide comparisons, and specificity was determined with a stringent yet adaptable calculation highlighting sera with outlying reactivity in an antigen-specific manner.

To compare ELISA and seromics results, we used a series of human sera known for their specific reactivity to individual antigens from our ELISA panel and tested them for reactivity with customized arrays. Results showed a high degree of similarity between the two methods, and all of the reactivities expected from ELISA were also observed in seromics. Overall, there was an excellent correlation (P<0.0001, Pearson correlation test) between the strength of antibody responses in ELISA as measured by titers, compared to seromics measured by foldover-cutoff results, suggesting that signal intensity in protein arrays may reflect relative actual titers.

### Example 4

### Discovery of Targets of Autoantibody Responses in Ovarian Cancer

A comparison of 52 sera from ovarian cancer patients with 53 sera from age-matched healthy donors was performed. (Note: control cohort was not sex-matched). For each antigen the sera that react significantly were determined.

The data for each microarray were normalized by a combination of transformations (expressed data for each array as ratio of interquartile difference, then performed quantile normalization on all arrays). Stringent cutoff for seroreactivity was determined based on interquartile differences of all data for each antigen. Final score includes: percentage of patient sera and healthy sera reacting (above cutoff) with each antigen, extent of reactivity observed (score defined as number of fold over cutoff), and compounded final score based on differential reactivity in patients compared to healthy sera. Only antigens with final compounded scores >5% (from either mean or median values) were considered to have significantly higher and/or stronger reactivity in cancer patients compared to healthy donors. Top list of antigens has 197 proteins considered specifically immunogenic in ovarian cancer patients. (see Table 3). Some of these proteins have already been shown to elicit spontaneous antibody responses in ovarian cancer (e.g., TP53) as well as in other cancer types, which further validates the effectiveness of the data analysis. Some antigens appear several times in the top list, indicating that different transcript variants of the same genes were found to have overlapping seroreactivity. There are many previously undescribed antigens (C.orf.) in the list as well as a few hypothetical proteins. Some of the known functions of immunogenic antigens appear to be related to cancer.

Following normalization and validation, individual antigens from microarrays were ranked according to the frequency of cancer sera reacting in comparison with the healthy cohort as well as by the mean strength of signal elicited. Contrary to gene array studies aiming at discovering small but consistent changes between two cohorts, the method used for seromics was designed to identify rare but clear events, corresponding to antigens only occasionally recognized with high titers among sera tested. To be considered of interest, antigen-specific responses had to occur more frequently in cancer patient sera than in healthy donor sera and be found in at least two patients within the cancer cohort, thus possibly representing shared tumor-associated events. Approximately 20% of all antigens failed to specifically react with any sera, and another 30% reacted with a single serum only. Out of all 8277 antigens, ovarian cancer sera reacted on average with 218 antigens (standard deviation=92) and healthy sera with 250 antigens (SD=121). Antigens eliciting responses with similar frequency and strength in patients and healthy donors failed to achieve a sufficient score to qualify as top antigens, and were thus not considered in the present analysis (even though some may represent important targets of autoimmunity or cancer immunosurveillance).

We found 197 distinct proteins with increased immunogenicity in ovarian cancer patients (Table 3). For 5 of these proteins (APEX1, CSNK2A1, GAS7, MAPKAPK5, SUB1), there were redundant sequences present on the array because of transcript variants produced independently, which also reacted with similar sets of sera, thereby confirming antigen specificity and bringing the total of frequently immunogenic gene products in ovarian cancer to 202. We observed that, although some occasional reactivity is observed in healthy donors, these antigens react preferentially and more strongly with sera from cancer patients.

The antigen with the highest score in ovarian cancer was UBTD2, also known as DC-UbP: It was immunogenic in 24% of patients, with an average reactivity of 4× over the cutoff, as compared to 6% of healthy donors reacting against it with less than 2× over the cutoff. Most other top antigens were recognized by less than 14% of patient serum samples, with a median differential frequency of 6%, and therefore represented rare events.

### Example 5

### Specificity Confirmation and Gene Ontology

A total of 19/197 (10%) antigens immunogenic by seromics in ovarian cancer patients have been previously identified by serological screening of cDNA expression libraries from various other cancer types (ALB, ANXA2, BRWD1, CBX5, CDC42EP2, CDC42EP4, CREB3L4, EFHD2, FER, FXYD5, GTF2I, MAGEA4, MCM7, MKNK2, NY-SAR-48, POLR3D, RPA2, RPS15A, and TP53), thereby confirming their immunogenic potential. For example, antigens ANXA2 or DNAJB1 were previously found to elicit autoantibodies in non-small-cell lung cancer.

Additionally, one target of ovarian cancer sera (MAPKAPK3) was recently identified as an immunoreactive antigen in colorectal cancer in one of the only other studies that used a similar strategy with ProtoArrays in a smaller sample set. Additionally, several top antigens immunogenic in ovarian cancer have been previously described associated with germ cells, oocyte maturation, or gonadal tumorigenesis, thus suggesting that humoral responses detected in seromics had specificity against the tumor type. More generally, a large number of top antigens have been found overexpressed in various cancer tissues, including pancreatic and ovarian, or associated with carcinogenesis. Yet, a majority of other proteins from the top list as well as previously unknown proteins, only discovered through domain homology or sequencing, have no assigned function or description of cancer association in the literature.

In an attempt to categorize top antigens according to biological pathways, we performed a gene ontology analysis of the top immunogenic candidate antigens in ovarian cancer. We were not able to define a unique functional or structural signature associated with candidate molecules, rather these genes belonged to many different pathways without Obvious connection to each other.

### Example 6

### Clinical Implications

Finally, we asked whether any of the top immunogenic antigens had a prognostic value for cancer survival. A clear limitation for such analyses lies in the low frequency of antibody responses observed, as well as in the heterogeneity of prior treatments as well as in stage heterogeneity, making statistical estimations difficult. To address this situation, an exploratory approach was followed. Survival analyses stratified for antibody responses against single antigens showed marked differences (FIG. 1), and individual antigens were found to be more often immunogenic in the serum of ovarian cancer patients with either favorable or bad prognosis (FIG. 1).

As an exploratory analysis and as a basis for further studies, potential good or bad prognostic antibody responses were determined using antigens in combination. FIG. 2 shows examples of markers with potential prognostic value. Because patient cohorts were selected on the basis of clinical outcome, these markers will need to be validated independently in a larger number of sera, once they are available as recombinant antigens for high-throughput screening. These exploratory observations give a first impression of the possible importance of antibody responses and show that antibody responses could indicate either a positive or negative clinical course.

Parts of the instant disclosure have been published in Gnjatic et al., Seromic profiling of ovarian and pancreatic cancer, PNAS 2010 107 (11) 5088-5093, incorporated herein by reference for disclosure of seromic profiling of ovarian cancer, measuring autoantibodies to known tumor antigens by ELISA, validating the use of protein microarrays, discovery of targets of autoantibody responses in ovarian and pancreatic cancer, specificity confirmation and gene ontology, and clinical implications.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the scope of the invention. Accordingly, the foregoing description is by way of example only. All references described herein are incorporated by reference for the purposes described herein. All sequences identified by the accession numbers in Table 3 herein are incorporated by reference for the purposes described herein.

Moreover, this invention is not limited in its application to the details of construction and the arrangement of components set forth in the disclosed description. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. A method for diagnosing ovarian cancer in a human comprising:
contacting a biological sample obtained from the human with at least one polypeptide selected from the polypeptides encoded by the transcripts disclosed in Table 3; and
determining an amount of specific binding between the at least one polypeptide and at least one antibody in the biological sample, wherein the amount of specific binding is diagnostic for ovarian cancer in the human.

2.-16. (canceled)

17. A method for diagnosis of ovarian cancer in a subject, comprising
(a) obtaining a biological sample from the subject;
(b) contacting the biological sample with a polypeptide selected from the polypeptides encoded by a transcript disclosed in Table 3, or with an antibody-binding fragment of the polypeptide; and
(c) determining the absence or the presence of an autoantibody specifically binding the polypeptide in the biological sample; wherein
(i) the presence of an autoantibody specifically binding a peptide encoded by a transcript disclosed in Table 3 in the biological sample is indicative of the subject having ovarian cancer,
(ii) the absence of an autoantibody specifically binding a peptide encoded by a transcript disclosed in Table 3 in the biological sample is indicative of the subject not having ovarian cancer; and/or
(d) determining a level of an autoantibody specifically binding the polypeptide in the biological sample and comparing the level to a reference or control level, wherein,
(i) an elevated level of an autoantibody specifically binding a peptide encoded by a transcript disclosed in Table 3 in the biological sample as compared to the reference or control level is indicative of the subject having ovarian cancer,
(ii) a non-elevated level of an autoantibody specifically binding a peptide encoded by a transcript disclosed in Table 3 in the biological sample as compared to the reference or control level is indicative of the subject not having ovarian cancer.

18. The method of claim 17, wherein the presence or an elevated level of an autoantibody specifically binding a peptide encoded by a transcript selected from the group consisting of ANXA2, FAM1318, FER, ZIM2, and NY-ESO1 transcripts is indicative of an increased expected survival time of the subject as compared to the average survival time of subjects having ovarian cancer or to the average survival time of ovarian cancer subjects in which the autoantibody is absent.

19. The method of claim 18, wherein the presence or elevated levels of autoantibodies specifically binding peptides encoded by ANXA2, FAM1318, FER, and ZIM2 transcripts is/are indicative of an increased expected survival time of the patient as compared to the average survival time of ovarian cancer patients or to the average survival time of ovarian cancer subjects in which the autoantibody is absent.

20. The method of claim 17, wherein the presence or an elevated level of an autoantibody specifically binding a peptide encoded by a transcript selected from the group consisting of ERFI1, PHLDB1, TRH, TRUB1, UBTD2 (DC-UbP) transcripts is indicative of a decreased expected survival time of the patient as compared to the average survival time of ovarian cancer patients or to the average survival time of ovarian cancer subjects in which the autoantibody is absent.

21. The method of claim 20, wherein the presence or elevated levels of autoantibodies specifically binding peptides encoded by ERFI1, PHLDB1, TRH, and TRUB1 transcripts is/are indicative of a decreased expected survival time of the patient as compared to the average survival time of ovarian cancer patients or to the average survival time of ovarian cancer subjects in which the autoantibody is absent.

22. The method of claim 17, wherein the biological sample is a blood sample.

23. The method of claim 17, wherein determining the presence or the absence of the autoantibody comprises performing an enzyme-linked immunoassay (ELISA).

24. The method of claim 17, wherein the polypeptide is fixed to a solid substrate.

25. The method of claim 24, wherein the solid substrate forms or is comprised in a plate well, and, optionally, wherein the plate well is in a multi-well plate optionally having a number of wells selected from the group consisting of: 6, 12, 24, 96, 384, and 1536.

26. The method of claim 24, wherein the solid substrate is a polypeptide array surface.

27. The method of claim 17, wherein determining the presence or absence of the autoantibody comprises contacting the autoantibody with a detection agent.

28. The method of claim 27, wherein the detection agent is a secondary antibody or antibody fragment, or an autoantibody-binding polypeptide conjugated to a detectable label.

29. The method of claim 27, wherein the detectable label is selected from the group consisting of: a radioisotope, a fluorophore, a luminescent molecule, an enzyme, a biotin-moiety, an epitope tag, and a dye molecule.

30. The method of claim 29, wherein the detectable label is a phosphatase, a peroxidase, an enzyme that catalyzes a chemical or biochemical reaction resulting in luminescence, or a fluorophore selected from the group comprising FITC, TRITC, Cy3, Cy5, Alexa Fluorescent Dyes, and Quantum Dots.

31. The method of claim 17, wherein determining the absence or the presence of an autoantibody specifically binding the polypeptide in the biological sample comprises determining a level of the autoantibody in the biological sample,
wherein, if the level of autoantibody specifically binding the polypeptide is greater than a cutoff level, then the autoantibody is present, and/or
wherein, if the level of autoantibody specifically binding the polypeptide is lower than a cutoff level, then the autoantibody is absent,

32. The method of claim 31, wherein
wherein, if the level of autoantibody specifically binding the polypeptide is greater than 2.5× the interquartile difference above the 75^{th} percentile of all autoantibody levels determined in the biological sample, then the autoantibody is present, and/or
wherein, if the level of autoantibody specifically binding the polypeptide is lower than 2.5× the interquartile difference above the 75^{th} percentile of all autoantibody levels determined in the biological sample, then the autoantibody is absent,

33. A method for prognosing patient outcome in ovarian cancer, the method comprising
determining the presence or a level of an autoantibody specifically binding a peptide encoded by a transcript selected from the group consisting of ANXA2, FAM1318, FER, ZIM2, NY-ESO1, ERFI1, PHLDB1, TRH, TRUB1, and UBTD2 (DC-UbP) transcripts in a subject having ovarian cancer,
wherein the presence of an autoantibody specifically binding a peptide encoded by an ANXA2, FAM1318, FER, ZIM2, or NY-ESO1 transcript or an elevated level of said autoantibody as compared to a reference or control level is indicative of a better patient outcome as compared to the average patient outcome of ovarian cancer patients, and/or
wherein the presence of an autoantibody specifically binding a peptide encoded by a transcript chosen from the group consisting of ERFI1, PHLDB1, TRH, TRUB1, UBTD2 (DC-UbP) transcripts or an elevated level of said autoantibody as compared to a reference or control level is indicative of a worse patient outcome as compared to the average survival time of ovarian cancer patients.

34. The method of claim 33,
wherein the presence of an autoantibody specifically binding a peptide encoded by an ANXA2, FAM1318, FER, ZIM2, or NY-ESO1 transcript or an elevated level of said autoantibody as compared to a reference or control level is indicative of an increased expected survival time of the patient as compared to the average survival time of ovarian cancer patients, and/or
wherein the presence of an autoantibody specifically binding a peptide encoded by a transcript chosen from the group consisting of ERFI1, PHLDB1, TRH, TRUB1, UBTD2 (DC-UbP) transcripts or an elevated level of said autoantibody as compared to a reference or control level is indicative of a decreased expected survival time of the patient as compared to the average survival time of ovarian cancer patients.

35. (canceled)

36. (canceled)

37. The method of claim 33, wherein determining the presence or a level of an autoantibody comprises obtaining a biological sample from the subject and contacting the biological sample with a polypeptide encoded by a transcript selected from the group consisting of ANXA2, FAM1318, FER, ZIM2, NY-ESO1, ERFI1, PHLDB1, TRH, TRUB1, and UBTD2 (DC-UbP) transcripts, or an antibody-binding fragment of said polypeptide

38.-70. (canceled)
